(19) United States
(12) Patent Application Publication
(10) Pub. No.: US 2024/0058477 A1
(43) Pub. Date: Feb. 22, 2024

(54) METHODS AND COMPOSITIONS FOR TREATMENT OF FRIEDREICH'S ATAXIA

(71) Applicants: University of Florida Research Foundation, Incorporated, Gainesville, FL (US); AavantiBio, Inc., Charlestown, MA (US)

(72) Inventors: Manuela Corti, Gainesville, FL (US); Barry John Byrne, Gainesville, FL (US); Irene Zolotukhin, Gainesville, FL (US)

(73) Assignees: University of Florida Research Foundation, Incorporated, Gainesville, FL (US); AavantiBio, Inc., Charlestown, MA (US)

(21) Appl. No.: 18/270,498

(22) PCT Filed: Jan. 4, 2022
(86) PCT No.: PCT/US2022/011189

§ 371 Date: Jun. 30, 2023

Related U.S. Application Data
(60) Provisional application No. 63/133,624, filed on Jan. 4, 2021.

(51) Int. Cl. A61K 48/00; A61K 45/06; A61P 25/00; C07K 14/47; C12N 15/86

(52) U.S. Cl. CPC A61K 48/0066; A61K 45/06; A61P 25/00; C07K 14/47; C12N 15/86; C12N 2750/14143; C12N 2800/22

(57) ABSTRACT

Provided for herein are polynucleotides, including codon-optimized polynucleotides, encoding genes for use in, for example, virally-mediated gene therapy for Friedreich's ataxia. Some embodiments related to viral vector constructs for use in such gene therapy. Also provided are dosing regimens and therapeutic combinations or systems for use in modulating immune responses to such viral vectors.

### CROSS-REFERENCE TO RELATED APPLICATIONS

This application claims priority under 35 U.S.C. § 119(e) to U.S. Provisional Patent Application No. 63/133,624, filed Jan. 4, 2021, which is hereby incorporated by reference in its entirety.

### FIELD

The present application relates generally to the field of gene therapy. More particularly, this application relates to delivery of a gene, or genes, including genes that are optimized in one or more manners, for the treatment of Friedreich's Ataxia.

### BACKGROUND

Friedreich's ataxia (“FA”) is a rare inherited disease that causes progressive nervous system damage and movement problems. In Friedreich's ataxia nerve fibers in the spinal cord and peripheral nerves degenerate, becoming thinner. It usually begins in childhood and leads to impaired muscle coordination (ataxia) that worsens over time. Effective therapies to treat FA are needed.

### INCORPORATION BY REFERENCE OF MATERIAL IN ASCII TEXT FILE

This application incorporates by reference the Sequence Listing contained in the following ASCII text file being submitted concurrently herewith: File Name: U120270078WO00-SEQ-EPG.txt; created Dec. 31, 2021, 25,629 bytes in size.

### SUMMARY

In view of the need for effective therapy for Friedreich's Ataxia, there is provided for herein, in several embodiments, methods and compositions for such therapy. In several embodiments, the methods and compositions provided for herein are improved as compared to other approaches, including other gene therapy approaches. In several embodiments, there is provided a polynucleotide encoding a codon optimized human frataxin, comprising a DNA sequence that has been codon optimized for enhanced expression and/or function in human cells having a sequence that comprises at least 85%, at least 90%, at least 95%, at least 97%, at least 98%, at least 99%, or more sequence identity to SEQ ID NO: 1. In several embodiments, the polynucleotide comprises a sequence having at least 99%, or more sequence identity to SEQ ID NO: 1.

In several embodiments, there is provided a polynucleotide encoding a codon optimized human frataxin, comprising a DNA sequence that has been codon optimized for enhanced expression and/or function having a sequence that comprises at least 85%, at least 90%, at least 95%, at least 97%, at least 98%, at least 99%, or more sequence identity to SEQ ID NO: 2. In several embodiments, the polynucleotide has a sequence having at least 99%, or more sequence identity to SEQ ID NO: 2. In some embodiments, the polynucleotide has a sequence that differs from either of SEQ ID NOs: 1 and 2 by 1, 2, 3, 4, 5, 6, 7, 8, 9, 12, 15 or more than 15 nucleotides.

In various embodiments, the polynucleotide has been codon optimized for expression in the cells of a particular species, such as a particular mammalian species. In various embodiments, the polynucleotide is codon optimized for expression in human cells. In several embodiments, the polynucleotide encodes human frataxin comprising the amino acid sequence of SEQ ID NO: 3.

In several embodiments, the polynucleotide exhibits enhanced expression of human frataxin as compared to a polynucleotide encoding frataxin but not having the same sequence identity to SEQ ID NO: 1. In several embodiments, there are only several nucleotides (e.g., 1, 2, 3, 4, 5, 5-10, 10-15, or 15-20 nucleotides) that differ between the provided polynucleotide and other frataxin transgenes for use in gene therapy. In some aspects, the provided polynucleotides represent further codon optimizations that advantageously result in improved expression and or function, and thus, more effective therapy.

In several embodiments, the polynucleotide further comprises an untranslated region that imparts regulatory control on expression of the amino acid sequence encoded by the polynucleotide. In some embodiments, the polynucleotide further comprises an untranslated region that imparts regulatory control on expression of the human frataxin encoded by the polynucleotide. In several embodiments, the untranslated region has a sequence that has at least 85%, at least 90%, at least 95%, at least 97%, at least 98%, at least 99%, or more sequence identity to SEQ ID NO: 6. In several embodiments, the untranslated region has a sequence that has at least 85%, at least 90%, at least 95%, at least 97%, at least 98%, at least 99%, or more sequence identity to SEQ ID NO: 12.

In several embodiments, the polynucleotide further comprises a promoter driving expression of the frataxin. In several embodiments, the promoter comprises a cytomegalovirus enhancer element functionally coupled to a chicken beta actin promoter. In several embodiments, the cytomegalovirus enhancer element has a sequence that comprises at least 85%, at least 90%, at least 95%, at least 97%, at least 98%, at least 99%, or more sequence identity to SEQ ID NO: 4. In several embodiments, the chicken beta actin promoter has a sequence that comprises at least 85%, at least 90%, at least 95%, at least 97%, at least 98%, at least 99%, or more sequence identity to SEQ ID NO: 5.

In several embodiments, the polynucleotides provided for herein are incorporated into a plasmid, e.g., a packaging plasmid. In several embodiments, the plasmid is at least 90% identical to SEQ ID NO: 8. In several embodiments, the plasmid is transfected into a packaging cell line, such as HEK293 cells in conjunction with one or more additional plasmids that encode viral packaging (e.g., rep and cap proteins) and viral particles are produced. In several embodiments, the polynucleotide is cloned into a non-therapeutic virus, such as a herpes simplex virus, which is used to infect a packaging cell line (along with another virus or plasmid encoding viral packaging proteins, e.g., rep and cap).

In accordance with several embodiments, there is also provided a recombinant adeno-associated virus (rAAV) vector for use in treating Friedreich's ataxia, wherein the vector comprises an expression construct comprising a promoter, a transgene encoding human frataxin, and a 3′ untranslated region, wherein the transgene encoding human frataxin comprises a DNA sequence that has been codon optimized for enhanced expression and/or function and having a sequence that comprises at least 85%, at least 90%, at least 95%, at least 97%, at least 98%, at least 99%, or more sequence identity to SEQ ID NO: 1. In several embodiments, the rAAV is serotype 9. In several embodiments, the rAAV is serotype rh74. In several embodiments, the rAAV comprises one or more of any of the polynucleotides disclosed herein.

In several embodiments, there is provided a system for treating Friedreich's Ataxia comprising an rAAV vector according to according to the present disclosure and an immunomodulatory regimen. In several embodiments, the immunomodulatory regimen comprises an antibody directed against a cancer marker and an additional agent. In several embodiments, the antibody is a full length antibody. In several embodiments, an scFv is used. In several embodiments, the cancer marker is CD20. In several embodiments, the antibody functions to reduce B-cell numbers and/or function. In several embodiments, the additional agent is a mTOR pathway inhibitor. In several embodiments, the additional agent blocks or disrupts signaling through the FKBP12 receptor. The mTOR inhibitor sirolimus is used as the additional agent, in several embodiments.

Further provided for herein is a dosing regimen for treatment of Friedreich's Ataxia, wherein the regimen comprises two doses: a first dose for intravenous delivery, and a second dose for delivery to cerebrospinal fluid (e.g., intrathecal delivery). In several embodiments, the viral genome content (or “amount”, in viral genomes per kg) of the first and second dose are within about 1 to about 20 times one another. In some embodiments, the viral genome content of the first dose is between 1 and 20 times as large as the viral genome content of the second dose. In other embodiments, the viral genome content of the second dose is between 1 and 20 times as large as the content of the first dose. In several embodiments, the first dose for intravenous delivery ranges from about 1×10¹³ to about 9×10¹⁵ viral genomes per kg body weight of a subject, and the second dose for delivery to cerebrospinal fluid ranges from about 9×10¹³ to about 9×10¹⁵ viral genomes per kg brain weight of the subject. In several embodiments, the second dose is delivered intrathecally.

In several embodiments, the dosing regimen further comprises an immunomodulatory regimen. In several embodiments, the immunomodulatory regimen comprises an antibody directed against a cancer marker and an additional agent. In several embodiments, the antibody is a full length antibody. In several embodiments, an scFv is used. In several embodiments, the cancer marker is CD20. In several embodiments, the antibody functions to reduce B-cell numbers and/or function. In several embodiments, the additional agent is a mTOR pathway inhibitor. In several embodiments, the additional agent blocks or disrupts signaling through the FKBP12 receptor. Sirolimus is used as the additional agent, in several embodiments.

In several embodiments, there is provided a method of treating Friedreich's Ataxia in a subject in need thereof, comprising administering to the subject a first dose of an rAAV vector encoding codon optimized human frataxin, wherein the codon optimized human frataxin comprises a DNA sequence that has been codon optimized for enhanced expression and/or function having a sequence that comprises at least 85%, at least 90%, at least 95%, at least 97%, at least 98%, at least 99%, or more sequence identity to SEQ ID NO: 1, wherein the first administration is intravenous, and administering to the subject a second dose of the rAAV vector encoding the codon optimized human frataxin, and wherein the second administration is to the cerebrospinal fluid. In several embodiments, the rAAV vector is serotype 9 or rh74. In several embodiments, the method further comprises administration of an immunomodulatory regimen comprising an antibody directed against a cancer marker and an additional agent. In several embodiments, the immunomodulatory regimen comprises an antibody directed against a cancer marker and an additional agent. In several embodiments, the antibody is a full length antibody. In several embodiments, an scFv is used. In several embodiments, the cancer marker is CD20. In several embodiments, the antibody functions to reduce B-cell numbers and/or function. In several embodiments, the additional agent is a mTOR pathway inhibitor. In several embodiments, the additional agent blocks or disrupts signaling through the FKBP12 receptor. Sirolimus is used as the additional agent, in several embodiments. In several embodiments, the immunomodulatory regimen is administered prior to the rAAV. In several embodiments, the immunomodulatory regimen is also administered after the rAAV. In several embodiments, the immunomodulatory regimen is administered before and/or after a subsequent dose or doses of rAAV (which need not be the same serotype as prior doses). In some embodiments, the subject is a human.

In some aspects, there is provided a use of the disclosed polynucleotides, plasmids, rAAVs, systems, or dosing regimens for treating Friedreich's ataxia. In several embodiments, there is provided a use of the disclosed polynucleotides, plasmids, rAAVs, systems, or dosing regimens for manufacture of a medicament for treating Friedreich's ataxia. In some aspects, there is provided any of the disclosed polynucleotides, plasmids, rAAVs, systems, or dosing regimens for use in the treatment or amelioration of Friedreich's ataxia, wherein the rAAV particle or composition is administered in a therapeutic amount to a subject having Friedreich's ataxia.

### BRIEF DESCRIPTION OF THE DRAWINGS

FIG. 1 depicts a non-limiting embodiment of a plasmid for use in generating exemplary viral vectors for treating Friedreich's ataxia.

FIG. 2 shows a study design for assessing the ability of frataxin vectors comprising SEQ ID NO: 11 (“FXN01”) as disclosed herein to restore physiological function in a mouse model.

FIG. 3 shows survival data (n=8 per group) with respect to the various treatment groups from the study design of FIG. 2.

FIGS. 4A and 4B show longitudinal body weight data. FIG. 4A shows body weight of male mice in the indicated groups over 22 weeks. FIG. 4B shows corresponding data for female mice.

FIGS. 5A and 5B show data (n=8 per group) and statistics for cardiac ejection fraction at 6, 10, and 13 weeks for the indicated experimental groups.

FIGS. 6A and 6B show data for and statistics cardiac fractional shortening at 6, 10, and 13 weeks for the indicated experimental groups.

FIGS. 7A and 7B show data and statistics for left ventricular mass at 6, 10, and 13 weeks for the indicated experimental groups.

FIGS. 8A and 8B show data and statistics for heart rate at 6, 10, and 13 weeks for the indicated experimental groups.

FIGS. 9A-9D show data and statistics for left ventricular anterior wall thickness during diastole (9A) and systole (9B).

FIGS. 10A-10D show data and statistics for left ventricular posterior wall thickness during diastole (10A) and systole (10B).

FIGS. 11A-11D show data and statistics for left ventricular inner diameter during diastole (11A) and systole (11B).

FIGS. 12A-12D show data for succinate dehydrogenase (SDH) staining, representative of activity, in cardiac tissues. FIG. 12A shows data related to the percentage of high SDH staining as a function of the area analyzed and FIG. 12C shows data related to the percentage of low SDH staining as a function of the area analyzed.

FIGS. 13A-13C illustrate data related to tissue biodistribution. FIG. 13A shows data related to the expression of frataxin in the heart for the indicated treatments. FIG. 13B shows data related to the expression of frataxin in the cerebellum for the indicated treatments. FIG. 13C shows information for the dosing for each delivery route.

FIGS. 14A and 14B show data related to the expression of frataxin in rat hearts 90 days post-injection. FIG. 14A shows a western blot of frataxin expression for the indicated treatment groups.

FIG. 14B shows information for the dosing for each delivery route.

FIGS. 15A-15C show data related to the processing of frataxin in the heart of non-human primates. FIG. 15A shows data from a serial dilution of cardiac tissue lysate from treated and control non-human primates, 90-days post administration of an rAAV-FXN vector. FIGS. 15B and 15C shows protein detection data (detected by chemiluminescence) from a serial dilution of cardiac tissue lysate from treated (15B) and control (15C) non-human primates, 90-days post administration of an rAAV-FXN vector.

FIGS. 16A-16C show data related to frataxin expression based on genes/vectors disclosed herein. FIG. 16A shows expression data for full-length (1), intermediate (2), and mature (3) frataxin after transfection into HEK293 cells. FIG. 16B shows data for expression of human frataxin from cells infected with a recombinant AAV generated through a herpes simplex virus production method. FIG. 16C shows chemiluminescence data for human frataxin expression after rAAV-hFXN infection of mouse tibialis anterior muscle and the associated western blot data.

FIGS. 17A and 17B show data related to the limited number of histopathological events detected in the dorsal root ganglia or spinal cord of non-human primates. FIG. 17A is a histogram showing the limited adverse pathology. FIG. 17B shows dosing information.

FIGS. 18A-18B show data related to immunomodulation and its impact on liver enzymes.

FIG. 18A shows data related to the detection of aspartate transaminase (AST) in the blood of non-human primates after receiving high-dose rAAV-hFXN either with, or without, a non-limiting immunosuppression regimen. FIG. 18B shows data related to the detection of alanine aminotransferase (ALT) in the blood of non-human primates after receiving high-dose rAAV-hFXN either with, or without, a non-limiting immunosuppression regimen.

FIG. 19 shows data related to frataxin expression based on genes/vectors disclosed herein. The Figure provides Western blot expression data showing full-length, intermediate, and mature frataxin after infection of HEK293 cells at various multiplicities of infection (MOI). Expression data following infection with rAAV comprising SEQ ID NO: 2 (improved codon-optimized human FXN) are those in lanes labeled “AVB-202,” while expression data following infection with rAAV comprising SEQ ID NO: 11 (“FXN01”) are those in lanes labeled “rAAV9-CBA-FXN01.”

FIG. 20 shows survival data (n=8 per group) with respect to the various treatment groups from the study utilizing rAAV comprising the improved codon-optimized FXN sequence (SEQ ID NO: 2).

FIG. 21 shows data and statistics for cardiac ejection fraction at 6, 10, 13, and 17 weeks for the indicated experimental groups from FIG. 20.

### DETAILED DESCRIPTION

The adeno-associated virus (AAV) genome comprises single-stranded deoxyribonucleic acid, which is about 4.7 kilobases long. The genome comprises inverted terminal repeats (ITRs) at both ends of the DNA strand, and two open reading frame, rep and cap. Rep is made up of four overlapping genes encoding Rep proteins required for the AAV life cycle, and cap contains overlapping nucleotide sequences of capsid proteins: VP1, VP2 and VP3, which interact to form a capsid

The ITR sequences are symmetrical and each comprise 145 nucleotides. The ITRs are able to form a hairpin, which contributes to self-priming allowing for primase-independent synthesis of the second DNA strand. The ITRs are required for both integration of the AAV DNA into the host cell genome and for efficient encapsidation of the AAV DNA combined with generation of a fully assembled, deoxyribonuclease-resistant AAV particles. ITRs are generally in cis next to the therapeutic transgene, for example in a plasmid, as discussed in greater detail below along with a promoter that drives expression of the transgene. Structural (cap) and packaging (rep) proteins can be delivered in trans. According to embodiments disclosed herein, AAV vectors are used to treat genetic disorders.

Friedreich's ataxia is caused by a defect (mutation) in the FXN gene, which carries the genetic code for a protein called frataxin. Individuals who inherit two defective copies of the gene, one from each parent, will develop the disease. Although rare, Friedreich's ataxia is the most common form of hereditary ataxia in the United States, affecting about 1 in every 50,000 people. There is currently no approved cure for Friedreich's ataxia.

The frataxin gene provides instructions for the production of a protein called frataxin. In the normal version of the gene, a triplet sequence of DNA (guanine-adenine-adenine, or GAA) is repeated between 7 and 22 times. In the defective frataxin gene, the GAA repeat occurs over and over again—hundreds, even up to a thousand times. This abnormal pattern is called a triplet repeat expansion, which greatly disrupts the normal production of frataxin. Earlier disease onset and severity of progression may be related to the number of GAA copies in the individual genetic code.

This triplet repeat expansion has been implicated as the cause of several diseases in which the individual needs to inherit only one abnormal gene. Friedreich's ataxia is the only known genetic disorder that requires inheriting two copies of the abnormal frataxin gene to cause the disease. 98% people with FA have two copies of this mutant form of frataxin. About two percent of affected individuals have other defects in the frataxin gene that are responsible for causing the disease.

Frataxin is a highly conserved, 210 amino acid (˜17 kDa) protein. It is found in the energy-producing parts of the cell called mitochondria. Research suggests that without a normal level of frataxin, certain cells in the body (especially peripheral nerve, spinal cord, brain and heart muscle cells) produce energy less effectively and have been hypothesized to have a buildup of toxic byproducts leading to what is called “oxidative stress.” Lack of normal levels of frataxin also may lead to increased levels of iron in the mitochondria. When the excess iron reacts with oxygen, free radicals can be produced. Although free radicals are essential molecules in the body metabolism, they can also destroy cells and harm the body. There is currently no approved cure for Friedreich's ataxia.

In some embodiments of the nucleic acid molecules and vectors provided herein, a transgene, or cDNA, encoding frataxin is about 630 nucleotides in length. In some embodiments, the transgene encoding frataxin is exactly 630 nucleotides in length.

Gene therapy (also called human gene transfer) is a medical field which focuses on the utilization of the therapeutic delivery of nucleic acids into a patient's cells as a drug to treat disease. The concept of gene therapy is to fix a genetic problem at its source. If, for instance, in an inherited disease a mutation in a certain gene results in the production of a dysfunctional protein, gene therapy could be used to deliver a copy of this gene that does not contain the deleterious mutation, and thereby produces a functional protein.

Several embodiments disclosed herein relate to addressing the multiorgan disease impact of FA, due to decreased level of frataxin in mitochondria (e.g., cardiomyopathy is primary cause of death and muscle control and coordination are affected). Several embodiments relate to AAV-mediated delivery of the frataxin gene. Several embodiments employ rAAV9, while others utilize other serotypes as disclosed herein. In several embodiments, the frataxin gene is targeted to cardiac and/or CNS tissues. In several embodiments, gene therapy is administered along with an immunomodulation strategy to mitigate and/or eliminate potential immune responses to rAAV vectors.

### Promoters

A “promoter”, as used herein, refers to a control region of a nucleic acid at which initiation and rate of transcription of the remainder of a nucleic acid sequence are controlled. A promoter drives transcription of the nucleic acid sequence that it regulates, thus, it is typically located at or near the transcriptional start site of a gene. A promoter may have, for example, a length of 100 to 1000 nucleotides. In some embodiments, a promoter is operably linked to a nucleic acid, or a sequence of a nucleic acid (nucleotide sequence). A promoter is considered to be “operably linked” to a sequence of nucleic acid that it regulates when the promoter is in a correct functional location and orientation relative to the sequence such that the promoter regulates (e.g., to control (or mediate, or drive) transcriptional initiation and/or expression of) that sequence.

Promoters that may be used in accordance with the present disclosure may comprise any promoter that can drive the expression of the transgenes in the heart of the subject. In some embodiments, the promoter may be an endogenous promoter (i.e., endogenous to the target cell) or an exogenous promoter promoter. In certain embodiments, the promoter is an endogenous promoter (e.g., an human frataxin promoter). In some embodiments, the promoter may be a constitutive or inducible promoter. In some embodiments, the promoter is a chicken β-actin (CBA) promoter, a hybrid CMV-CBA promoter, or a truncated CBA (smCBA) promoter. In some embodiments, the promoter may be a tissue-specific promoter. A “tissue-specific promoter”, as used herein, refers to promoters that can only function in a specific type of tissue, e.g., cardiac tissue. Thus, a “tissue-specific promoter” is not able to drive the expression of the transgenes in other types of tissues. In some embodiments, the promoter that may be used in accordance with the present disclosure is a cardiac-specific promoter. For example, promoter is a cardiac-restricted promoter selected from cardiac troponin C, cardiac troponin I, and cardiac troponin T (cTnT). In some embodiments, the promoter that may be used in accordance with the present disclosure is a muscle-specific promoter, such as desmin. In some embodiments, the promoter that may be used in accordance with the present disclosure is a CNS-specific promoter, such as the synapsin (SYN) promoter.

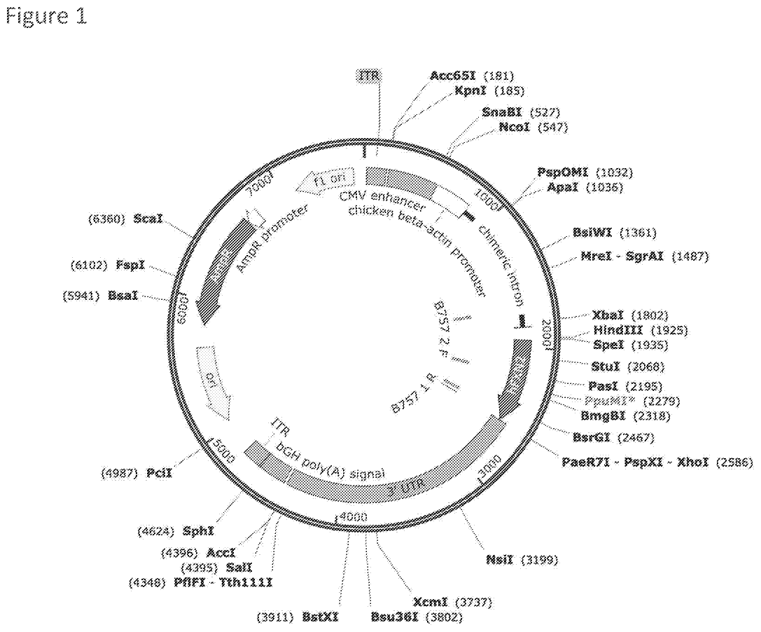

FIG. 1 represents a non-limiting embodiment of a plasmid encoding a transgene, which is used for production of an rAAV particle for use in gene therapy. As discussed above and as shown in FIG. 1, the plasmid comprises two ITRs that flank a transgene of interest, the expression of which is driven by a promoter. The promoter used in a given embodiment can be selected based on its ability, or strength, to yield sufficient and durable expression of the transgene.

In some embodiments, the promoter includes one or more of the following: a Desmin promoter, a chicken β-actin (CBA) promoter, or an endogenous human frataxin promoter (hFXNPro). In several embodiments, the CBA promoter is a hybrid promoter, consisting of the CMV enhancer and CBA promoter. Advantageously, this promoter directs durable expression of frataxin in target tissues relevant to FA including the heart, cerebellar neurons, and sensory neurons of the dorsal root ganglia. Moreover, in several embodiments, efficient promoter activity allows for a reduced vector dose to minimize direct capsid mediated toxicity. In several embodiments, the CMV enhancer is encoded by SEQ ID NO: 4, or a sequence having at least 85%, at least 90%, at least 95%, or at least 98% sequence identity with SEQ ID NO: 4. In several embodiments, the CBA promoter is encoded by SEQ ID NO: 5, or a sequence having at least 85%, at least 90%, at least 95%, or at least 98% sequence identity with SEQ ID NO: 5.

In some embodiments, the Desmin promoter is encoded by SEQ ID NO: 9, or a sequence having at least 85%, at least 90%, at least 95%, or at least 98% sequence identity with SEQ ID NO: 9. In some embodiments, the hFXNPro is encoded by SEQ ID NO: 10, or a sequence having at least 85%, at least 90%, at least 95%, or at least 98% sequence identity with SEQ ID NO: 10.

As a practical matter, whether any particular nucleic acid molecule is at least 80%, at least 85%, at least 90%, at least 95%, at least 96%, at least 97%, at least 98%, or at least 99% identical to, for instance, the nucleotide sequence of a transgene, can be determined conventionally using known computer programs. A preferred method for determining the best overall match between a query sequence (e.g., a sequence of the present disclosure) and a subject sequence, also referred to as a global sequence alignment, can be determined using the FASTDB or blastn computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. Whether a nucleotide is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present disclosure. For subject sequences truncated at the 5′ and/or 3′ termini, relative to the query sequence, the percent identity is corrected by calculating the number of nucleotides of the query sequence that are positioned 5′ to or 3′ to the query sequence, which are not matched/aligned with a corresponding subject nucleotide, as a percent of the total bases of the query sequence.

As used herein, the term “variant” refers to a nucleic acid having characteristics that deviate from a reference sequence (e.g., that which occurs in nature). For example, a “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to a reference sequence (e.g., the wild type nucleic acid). In some embodiments, the reference sequence may be the wild type or naturally occurring sequence corresponding to the variant. For instance, a transgene variant is a nucleic acid comprising one or more substitutions in the nucleotides of a transgene, as compared to the wild type sequence thereof. In some embodiments, the reference sequence may be a sequence which has been codon optimized, relative to the wild type or naturally occurring sequence, for expression in a particular subject or tissue, and which, in either the wild type or codon optimized configuration, corresponds to the variant. For instance, a transgene variant is a nucleic acid comprising one or more substitutions in the nucleotides of a transgene, as compared to the codon optimized sequence thereof. These substitutions may be silent; e.g., they do not modify the amino acid sequence of any encoded protein (or otherwise result in a variant amino acid sequence).

Alternatively, these substitutions may result in modifications to the amino acid sequence of an encoded protein, resulting in an encoded protein having one or more amino acid substitutions (e.g., having 1, 2, 3, 4, 5, 6, 7, 8, 9, 10, 10-15, or 15-20 amino acid substitutions) relative to the wild type protein sequence. These substitutions include chemical modifications as well as truncations. This term further embraces functional fragments of a wild type nucleic acid sequence. These modifications of the reference sequence may occur at the 5′ or 3′ ends of the reference sequence or anywhere between those positions, interspersed either individually among nucleotides in the reference sequence or in one or more contiguous groups within the reference sequence.

A “transgene”, as used herein, refers to a gene or genetic material that has been transferred naturally, or by any of a number of genetic engineering techniques from one organism to another. A transgene may be a protein or polypeptide of interest (e.g., human FXN) or an RNA of interest (e.g., a siRNA or shRNA). In some embodiments, any of the disclosed vectors, such as any of the disclosed viral vectors, may comprise the coding sequence for one or more transgenes. In some embodiments, the rAAV vectors of the present disclosure comprise the coding sequence of hFXN or variants thereof.

### Therapeutic Transgenes

In some embodiments, the principle of the AAV-based gene therapy methods and compositions disclosed herein is based on the ability to restore proper frataxin function in target tissues. Accordingly, in some embodiments, the rAAV particles and methods provided herein may restore normal frataxin protein activity in a subject, such as a human subject. Some embodiments relate to a nucleic acid that include an expression construct comprises a promoter, as discussed above, the transgene, and a 3′-untranslated region (3′-UTR). The expression construct contains a human FXN coding sequence, which is codon optimized (for expression in human cells) in several embodiments, encoded by SEQ ID NO: 1, or a or a sequence having at least 85%, at least 90%, at least 95%, or at least 98% sequence identity with SEQ ID NO: 1. In several embodiments, the frataxin transgene encodes the amino acid sequence of SEQ ID NO: 3, or an amino acid sequence having at least 85%, at least 90%, at least 95%, or at least 98% sequence identity of SEQ ID NO: 3. In several embodiments, the frataxin transgene encodes the amino acid sequence of a protein having at least 85%, at least 90%, at least 95%, or at least 98% of the function of the protein set forth in SEQ ID NO: 3 (frataxin). The sequence of SEQ ID NO: 3 is 210 amino acids in length.

In some embodiments, any of the disclosed polynyucleotides encode a frataxin protein having a sequence that differs from the sequence of SEQ ID NO: 3 by 1, 2, 3, 4, 5, 6, 7, 8, 9, 10, 11, 12, or more than 12 amino acids. In some embodiments, any of the disclosed polynucleotides encode a protein that is truncated by 1, 2, 3, or more than 3 amino acids at the N- or C-terminus relative to SEQ ID NO: 3. In some embodiments, any of the disclosed polynucleotides encode one or more protein sequences that have stretches of 50, 75, 90, 93, 100, 125, 175, 200, or more than 200 amino acids in common with SEQ ID NO: 3.

In several embodiments, the 3′-UTR is a native untranslated region of the frataxin gene. In several embodiments, the 3′-UTR is truncated as compared to a native 3′-UTR. In some embodiments, the truncated 3′-UTR is about 1400, 1450, 1480, 1490, 1500, 1550, 1600, 1700, 1750, 1800, 1815, 1830, 1850 or 1900 base pairs (bp) in length. In certain embodiments, the truncated 3′-UTR is 1490 bp in length. In certain embodiments, the truncated 3′-UTR is 1813 bp in length. In certain embodiments, the truncated 3′-UTR is encoded by SEQ ID NO: 6 (which is 1813 bp in length). In several embodiments, the truncated 3′-UTR is encoded by a sequence having at least 85%, at least 90%, at least 95%, or at least 98% sequence identity with SEQ ID NO: 6. In certain embodiments, the truncated 3′-UTR is 1490 bp in length. In certain embodiments, the truncated 3′-UTR is encoded by SEQ ID NO: 12 (which is 1490 bp in length). In several embodiments, the truncated 3′-UTR is encoded by a sequence having at least 85%, at least 90%, at least 95%, or at least 98% sequence identity with SEQ ID NO: 12. In some embodiments, the UTR is truncated at the 3′ terminus. In some embodiments, the UTR is truncated at the 5′ terminus. In some embodiments, the truncated 3′-UTR is about 2360 bp, 2260 bp, 2160 bp, 2060 bp, 1960 bp, 1860 bp, 1810 bp, 1760 bp, 1660 bp, 1560 bp, 1500 bp, 1490 bp, 1475 bp, 1460 bp, 1360 bp, 1260 bp, 1160 bp, 1060 bp, or 960 by in length.

In several embodiments, the truncated 3′-UTR is truncated by about 100, about 200, about 300, about 400, about 500, about 600, about 640, about 650, about 700, about 800, about 825, about 850, about 860, about 870, about 885, about 900, about 1000, about 1200, about 1400, about 1600, about 1800, or about 2000 nucleotides (or any amount between those values) as compared to a native (or wild-type) 3′-UTR. In some embodiments, the truncated 3′-UTR is truncated by 868 nucleotides as compared to a native 3′-UTR. In some embodiments, the truncated 3′-UTR is truncated by 645 nucleotides as compared to a native 3′-UTR. In some embodiments, the native 3′-UTR is 2458 bp in length. In some embodiments, the truncated 3′-UTR comprises a sequence that is between 100 and 1000, between 200 and 900, between 300 and 800, between 200 and 800, between 300 and 700, between 300 and 500, between 400 and 800, between 500 and 800, between 500 and 700, between 400 and 650, between 500 and 650, or between 500 and 600 nucleotides shorter than a wild-type human FXN 3′ UTR. In certain embodiments, the truncated 3′-UTR comprises a sequence that is between 500 and 800 nucleotides shorter than a wild-type human FXN 3′ UTR. In certain embodiments, the truncated 3′-UTR comprises a sequence that is between 500 and 650 nucleotides shorter than a wild-type human FXN 3′ UTR. In some embodiments, the native 3′-UTR is encoded by any one of several alternative transcript variants of native frataxin. In some embodiments, the native 3′-UTR is encoded by a native frataxin transcript variant having a sequence set forth as GenBank No. BCO23633. In other embodiments, the native 3′-UTR is encoded by a native frataxin transcript variant having a sequence is set forth as GenBank No. NM_00144.5. In some embodiments, the truncated 3′-UTR of the disclosed polynucleotides is truncated relative to the 3′-UTR of the native frataxin of GenBank No. BC023633 or GenBank No. NM_00144.5.

In several embodiments, the 3′-UTR confers post-transcriptional control on expression of frataxin, thus preventing potential toxicity resulting from overexpression of FXN. In several embodiments, the expression construct is contained in the plasmid encoded by SEQ ID NO: 8. In several embodiments, the expression construct is encoded by residues 185-2579 of SEQ ID NO: 8, or a sequence having at least 85%, at least 90%, at least 95%, or at least 98% sequence identity to SEQ ID NO: 8. In some embodiments, provided herein is an rAAV vector comprising a polynucleotide having at least 85%, at least 90%, at least 95%, or at least 98% sequence identity to SEQ ID NO: 8. In some embodiments, provided herein is an rAAV vector comprising a polynucleotide comprising the sequence of SEQ ID NO: 8.

In several embodiments, there is provided a polynucleotide encoding a codon optimized human frataxin, comprising a DNA sequence that has been codon optimized for enhanced expression and/or function having a sequence that comprises at least 85%, at least 90%, at least 95%, at least 97%, at least 98%, at least 99%, or more sequence identity to SEQ ID NO: 1 or SEQ ID NO: 2. In several embodiments, the polynucleotide has a sequence having at least 99%, or more sequence identity to SEQ ID NO: 1 or SEQ ID NO: 2. The sequence set forth as SEQ ID NO: 2 differs from that of SEQ ID NO: 1 in that a STOP codon is absent in SEQ ID NO: 2.

In some aspects, provided herein are polynucleotides and vectors (e.g., rAAV nucleic acid vectors) that comprise a sequence that is at least 90%, at least 95%, or at least 99.5% identical to any one of the nucleotide sequences of SEQ ID NOs: 1, 2, 8 and 11. In some embodiments, the polynucleotide sequence differs from the sequence of any one of SEQ ID NOs: 1, 2, 8 and 11 by 1, 2, 3, 4, 5, 6, 7, 8, 9, 10, 11, 12, or more than 12 nucleotides. In some embodiments, the polynucleotide sequence contains stretches of about 35, 50, 75, 100, 125, 150, 175, 200, 225, 240, 255, 275, 300, 350, 400 or more than 400 consecutive nucleotides in common with any one of SEQ ID NOs: 1, 2, 8 and 11. In particular embodiments, any of the disclosed rAAV vectors comprise a polynucleotide that comprises any of SEQ ID NOs: 1, 2, 8 and 11. In some aspects, provided herein are polynucleotides encoding a 3′-UTR that differs from the sequence of SEQ ID NO: 6 or 12 by 1, 2, 3, 4, 5, 6, 7, 8, 9, 10-15, 15-20, 20-25, or more than 25 nucleotides. In some embodiments, the disclosed polynucleotide sequence contains stretches of about 35, 50, 75, 100, 125, 150, 175, 200, 225, 240, 255, 275, 300, 350, 400, 500 or more than 500 consecutive nucleotides in common with SEQ ID NO: 6 or 12. In particular embodiments, any of the disclosed rAAV vectors comprise a polynucleotide that comprises either of SEQ ID NOs: 6 and 12.

Improved codon-optimized human frataxin coding sequence (with stop codon)
(SEQ ID NO: 1)
ATGTGGACCCTCGGCCGCAGAGCTGTTGCTGGACTTCTTGCCTCTCCATCTCCAGCTCAGGCCCAG
ACACTGACCAGAGTGCCTAGACCTGCTGAACTGGCCCCTCTGTGTGGCAGAAGAGGCCTGAGAACC
GACATCGACGCCACATGCACACCTAGAAGGGCCAGCAGCAATCAGCGGGGCCTGAATCAGATCTGG
AACGTGAAGAAACAGAGCGTGTACCTGATGAACCTGAGAAAGAGCGGCACCCTGGGACACCCTGGA
AGCCTGGATGAGACAACCTACGAGAGACTGGCCGAGGAAACCCTGGATTCCCTGGCCGAGTTCTTC
GAGGACCTGGCCGATAAGCCCTACACCTTCGAGGATTACGACGTGTCCTTTGGCAGCGGCGTGCTG
ACAGTGAAACTCGGCGGAGATCTGGGCACCTACGTGATCAACAAGCAGACCCCTAACAAACAGATC
TGGCTGAGCAGCCCTAGCAGCGGCCCCAAGAGATATGATTGGACCGGCAAGAACTGGGTGTACAG
CCACGATGGCGTGTCCCTGCACGAACTGCTGGCTGCCGAACTGACAAAGGCCCTGAAAACAAAGCT
GGACCTGTCCAGCCTGGCCTACTCTGGCAAGGACGCCTGA
Improved codon-optimized human frataxin coding sequence (without stop codon)
(SEQ ID NO: 2)
ATGTGGACCCTCGGCCGCAGAGCTGTTGCTGGACTTCTTGCCTCTCCATCTCCAGCTCAGGCCCAG
ACACTGACCAGAGTGCCTAGACCTGCTGAACTGGCCCCTCTGTGTGGCAGAAGAGGCCTGAGAACC
GACATCGACGCCACATGCACACCTAGAAGGGCCAGCAGCAATCAGCGGGGCCTGAATCAGATCTGG
AACGTGAAGAAACAGAGCGTGTACCTGATGAACCTGAGAAAGAGCGGCACCCTGGGACACCCTGGA
AGCCTGGATGAGACAACCTACGAGAGACTGGCCGAGGAAACCCTGGATTCCCTGGCCGAGTTCTTC
GAGGACCTGGCCGATAAGCCCTACACCTTCGAGGATTACGACGTGTCCTTTGGCAGCGGCGTGCTG
ACAGTGAAACTCGGCGGAGATCTGGGCACCTACGTGATCAACAAGCAGACCCCTAACAAACAGATC
TGGCTGAGCAGCCCTAGCAGCGGCCCCAAGAGATATGATTGGACCGGCAAGAACTGGGTGTACAG
CCACGATGGCGTGTCCCTGCACGAACTGCTGGCTGCCGAACTGACAAAGGCCCTGAAAACAAAGCT
GGACCTGTCCAGCCTGGCCTACTCTGGCAAGGACGCC
“FXN01” human frataxin coding sequence
(SEQ ID NO: 11)
ATGTGGACACTGGGGAGAAGGGCCGTGGCTGGACTGCTGGCTTCTCCATCTCCAGCCCAGGCCCA
GACCCTGACCAGAGTGCCTAGACCTGCCGAACTGGCCCCTCTGTGTGGCAGAAGAGGCCTGAGAA
CCGACATCGACGCCACCTGTACCCCCAGAAGGGCCAGCAGCAATCAGCGGGGCCTGAATCAGATCT
GGAACGTGAAGAAACAGAGCGTGTACCTGATGAACCTGAGAAAGAGCGGCACCCTGGGCCACCCT
GGAAGCCTGGATGAGACAACCTACGAGCGGCTGGCCGAGGAAACCCTGGATTCCCTGGCCGAGTT
CTTCGAGGACCTGGCCGACAAGCCCTACACCTTCGAGGATTACGACGTGTCCTTCGGCAGCGGCGT
GCTGACAGTGAAGCTGGGCGGAGATCTGGGCACCTACGTGATCAACAAGCAGACCCCCAACAAACA
GATCTGGCTGAGCAGCCCCAGCAGCGGCCCCAAGAGATACGATTGGACCGGCAAGAACTGGGTGT
TCAGCCACGACGGCGTGTCCCTGCATGAGCTGCTGGCTGCCGAGCTGACCAAGGCCCTGAAAACAA
AGCTGGACCTGAGCTGGCTGGCCTACAGCGGCAAAGATGCC
Exemplary truncated FXN 3′-UTR-coding sequence
(SEQ ID NO: 12)
AAGAAGGAAAAATTCCAGGAGGGAAAATGAATTGTCTTCACTCTTCATTCTTTGAAGGATTTACTGCA
AGAAGTACATGAAGAGCAGCTGGTCAACCTGCTCACTGTTCTATCTCCAAATGAGACACATTAAAGG
GTAGCCTACAAATGTTTTCAGGCTTCTTTCAAAGTGTAAGCACTTCTGAGCTCTTTAGCATTGAAGTG
TCGAAAGCAACTCACACGGGAAGATCATTTCTTATTTGTGCTCTGTGACTGCCAAGGTGTGGCCTGC
ACTGGGTTGTCCAGGGAGACATGCATCTAGTGCTGTTTCTCCCACATATTCACATACGTGTCTGTGT
GTATATATATTTTTTCAATTTAAAGGTTAGTATGGAATCAGCTGCTACAAGAATGCAAAAAATCTTCCA
AAGACAAGAAAAGAGGAAAAAAAGCCGTTTTCATGAGCTGAGTGATGTAGCGTAACAAACAAAATCA
TGGAGCTGAGGAGGTGCCTTGTAAACATGAAGGGGCAGATAAAGGAAGGAGATACTCATGTTGATA
AAGAGAGCCCTGGTCCTAGACATAGTTCAGCCACAAAGTAGTTGTCCCTTTGTGGACAAGTTTCCCA
AATTCCCTGGACCTCTGCTTCCCCATCTGTTAAATGAGAGAATAGAGTATGGTTGATTCCCAGCATTC
AGTGGTCCTGTCAAGCAACCTAACAGGCTAGTTCTAATTCCCTATTGGGTAGATGAGGGGATGACAA
AGAACAGTTTTTAAGCTATATAGGAAACATTGTTATTGGTGTTGCCCTATCGTGATTTCAGTTGAATTC
ATGTGAAAATAATAGCCATCCTTGGCCTGGCGCGGTGGCTCACACCTGTAATCCCAGCACTTTTGGA
GGCCAAGGTGGGTGGATCACCTGAGGTCAGGAGTTCAAGACCAGCCTGGCCAACATGATGAAACCC
CGTCTCTACTAAAAATACAAAAAATTAGCCGGGCATGATGGCAGGTGCCTGTAATCCCAGCTACTTG
GGAGGCTGAAGCGGAAGAATCGCTTGAACCCAGAGGTGGAGGTTGCAGTGAGCCGAGATCGTGCC
ATTGCACTGTAACCTGGGTGACTGAGCAAAACTCTGTCTCAAAATAATAATAACAATATAATAATAATA
ATAGCCATCCTTTATTGTACCCTTACTGGGTTAATCGTATTATACCACATTACCTCATTTTAATTTTTAC
TGACCTGCACTTTATACAAAGCAACAAGCCTCCAGGACATTAAAATTCATGCAAAGTTATGCTCATGT
TATATTATTTTCTTACTTAAAGAAGGATTTATTAGTGGCTGGGCATGGTGGCGTGCACCTGTAATCCC
AGGTACTCAGGAGGCTGAGACGGGAGAATTGCTTGACCCCAGGCGGAGGAGGTTACAGTGAGTCG
AGATCGTACCTGAGCGACAGAGCGAGACTCCGTCTCAAAAAAAAAAAAAAGGAGGGTTTATTAATGA
GAAGTTTG

### Vectors

Aspects of the present disclosure relate to recombinant AAV vectors that may be used for gene therapy for heart diseases. As used herein, the term “vector” may refer to a nucleic acid vector (e.g., a plasmid or recombinant viral genome), a wild-type AAV genome, or a virus that comprises a viral genome. In some embodiments, the term “vector” may refer to a viral particle, such as an AAV viral particle.

The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners. Either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. A mature AAV capsid is composed of VP1, VP2, and VP3 (molecular masses of approximately 87, 73, and 62 kDa respectively) in a ratio of about 1:1:10.

Recombinant AAV (rAAV) particles may comprise a recombinant nucleic acid vector (hereafter referred to as “rAAV vector”), which may comprise at a minimum: (a) one or more polynucleotides comprising a sequence encoding a transgene; and (b) one or more regions comprising sequences that facilitate the integration of the polynucleotide (optionally with the one or more nucleic acid regions comprising a sequence that facilitates expression) into the genome of the subject. In some embodiments, the sequences facilitating the integration of the polynucleotide (optionally with the one or more nucleic acid regions comprising a sequence that facilitates expression) into the genome of the subject are inverted terminal repeat (ITR) sequences (e.g., wild-type ITR sequences or engineered ITR sequences) flanking the one or more nucleic acid regions (e.g., polynucleotides).

In some embodiments, the rAAV vectors of the present disclosure further comprise an Internal Ribosome Entry Site (IRES). An IRES is a nucleotide sequence that allows for translation initiation in the middle of a messenger RNA (mRNA) sequence as part of the greater process of protein synthesis. Usually, in eukaryotes, translation can be initiated only at the 5′ end of the mRNA molecule, since 5′ cap recognition is required for the assembly of the initiation complex. In some embodiments, the IRES is located between the transgenes. In such embodiments, the proteins encoded by different transgenes are translated individually (i.e., versus translated as a fusion protein).

In some embodiments, the rAAV vectors of the present disclosure further comprise a polyadenylation (pA) signal. Eukaryotic mRNAs are typically transcribed as a precursor mRNA. The precursor mRNA is processed to generate the mature mRNA, including a polyadenylation process. The process of polyadenylation begins as the transcription of a gene terminates. The 3′-most segment of the newly made precursor mRNA is first cleaved off by a set of proteins. These proteins then synthesize the poly(A) tail at the RNA's 3′ end. The cleavage site typically contains the polyadenylation signal, e.g., AAUAAA. The poly(A) tail is important for the nuclear export, translation, and stability of mRNA.

In exemplary embodiments, the ITR sequences flank a nucleic acid region comprising two or more transgenes and a promoter operably linked to the two or more transgenes and a polyadenylation (polyA) signal, In exemplary embodiments, the ITR sequences flank a nucleic acid region consisting of two or more transgenes and a promoter operably linked to the two or more transgenes and a polyA signal,

In some embodiments, the polynucleotide of the rAAV nucleic acid vector comprises one or more transgenes comprising a sequence encoding a protein or polypeptide of interest operably linked to a promoter, wherein the one or more transgenes are flanked on each side with an ITR sequence. In some embodiments, the nucleic acid vector further comprises a region encoding a Rep protein as described herein, either contained within the region flanked by ITRs or outside the region or nucleic acid) operably linked to a promoter. The ITR sequences may be derived from any AAV serotype (e.g., 1, 2, 3, 4, 5, 6, 7, 8, 9, or 10) or may be derived from more than one serotype. In some embodiments, the ITR sequences are derived from AAV2 or AAV6 serotypes. In some embodiments, a first serotype provided herein is not an AAV2 or AAV8 serotype. In some embodiments, the ITR sequences of the first serotype are derived from AAV3, AAV5 or AAV6. In some embodiments, the ITR sequences are derived from AAV2, AAV3, AAV5 or AAV6. In some embodiments, the ITR sequences are the same serotype as the capsid (e.g., AAV6 ITR sequences and AAV6 capsid, etc.). In some embodiments, the ITR sequences are derived from AAVrh.10 serotype.

ITR sequences and plasmids containing ITR sequences are known in the art and commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, et al. Proc Natl Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, Samuel M. Young, Jr., Toni Cathomen and Richard Jude Samulski; U.S. Pat. Nos. 5,139,941 and 5,962,313, all of which are incorporated herein by reference). In some embodiments, the rAAV comprises a pTR-UF-11 plasmid backbone, which is a plasmid that contains AAV2 ITRs. This plasmid is commercially available from the American Type Culture Collection (ATCC MBA-331).

Thus, in some embodiments, the rAAV vector comprises one or more regions comprising a sequence that facilitates expression of the transgene, e.g., expression control sequences operably linked to the nucleic acid. Numerous such sequences are known in the art. Non-limiting examples of expression control sequences include promoters, insulators, silencers, response elements, introns, enhancers, initiation sites, internal ribosome entry sites (IRES) termination signals, and poly(A) signals. Any combination of such control sequences is contemplated herein (e.g., a promoter and a poly(A) signal). In some embodiments, the rAAV vectors comprise a promoter that is operably linked to the coding sequence of the transgenes and facilitates expression of the transgenes.

In some embodiments, the rAAV is circular. In some embodiments, the rAAV vector is linear. In some embodiments, the rAAV vector is single-stranded. In some embodiments, the rAAV vector is double-stranded. In some embodiments, the rAAV vector is a self-complementary rAAV vector. In some embodiments, the rAAV vector comprises the complement of any of the disclosed rAAV vector sequences.

The capsid protein of any of the AAV particles provided for herein, e.g., encapsidating any of the disclosed recombinant AAV vectors, can be of any serotype. For example, in several embodiments, AAV serotype 9 (AAV9) is used. In several embodiments, AAV1, AAV2, AAV4, AAV5, AAA6, AAV7, AAV8, AAV9, AAV10, AAV11, AAV-DJ, AAV rhesus 10 (rh.10), or rh74 (i.e., AAVrh.74). In several embodiments, AAV serotype rh74, or a variant thereof, is used. In some embodiments, rh74 AAV variant is used which is mutated to advantageously enhance delivery to cardiac tissue, for example by a tryptophan to arginine mutation at amino acid 505 of VP1 capsid (W505R), or other mutations, as described in PCT Publication WO 2019/1784412, which is incorporated in its entirety by reference herein. Pseudotyped vectors are also used in several embodiments, for example, AAV2/9 (or any pseudotyped combination of those listed herein). A pseudotyped rAAV particle, which comprises (a) an rAAV vector comprising ITRs from one serotype (e.g., AAV2, AAV3) and (b) a capsid comprised of capsid proteins derived from another serotype (e.g., AAV1, AAV2, AAV3, AAV4, AAV5, AAV6, AAV7, AAV8, AAV9, or AAV10). Such AAV serotypes and derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001). In some embodiments, the capsid is of the AAV2/9 serotype.

Some embodiments relate to a recombinant adeno-associated virus (rAAV) particle comprising the nucleic acids disclosed herein. In some embodiments, the rAAV particle is an AAV9 particle. Some embodiments relate to a composition comprising a plurality of the rAAV particles disclosed herein. In some embodiments, the composition further includes a pharmaceutically acceptable carrier. The term “carrier” refers to a diluent, adjuvant, excipient, or vehicle with which the rAAV particle or preparation, and/or rAAV vectors is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions may also be employed as liquid carriers. Formulations comprising pharmaceutically-acceptable excipients and/or carrier solutions are well-known to those of skill in the art, as is the development of suitable dosing and treatment regimens for using the particular compositions described herein in a variety of treatment regimens, including e.g., intravenous, intrathecal, intracerebroventricular, intra-arterial, subcutaneous, intramuscular, intrahepatic, intraperitoneal and/or local administration and formulation.

In several embodiments, wherein a plurality of doses of rAAV are administered, the first and any subsequent dose may be the same serotype, or of different serotypes. In several embodiments, a supporting therapy is administered in order to reduce or eliminate potential immune responses in a patient to a second or subsequent dose of rAAV-hFXN. Additional information related to rAAV vectors for treating FA can be found in U.S. Pat. No. 10,617,770, which published as U.S. Publication No. 2018/0117178 on May 3, 2018, which is incorporated in its entirety by reference herein.

Accordingly, in some aspects, further provided herein are rAAV particles or preparations containing such particles. The rAAV particles comprise a viral capsid and an rAAV vector as described herein, which is encapsidated by the viral capsid. Methods of producing rAAV particles are known in the art and are commercially available (see, e.g., Zolotukhin et al. Production and purification of serotype 1, 2, and 5 recombinant adeno-associated viral vectors. Methods 28 (2002) 158-167; and U.S. Patent Publication Nos. US 2007/0015238 and US 2012/0322861, which are incorporated herein by reference; and plasmids and kits available from ATCC and Cell Biolabs, Inc.). For example, a plasmid containing the rAAV vector may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified. In various embodiments, any of the disclosed rAAV particles are generated using a herpes simplex virus (HSV) production method which is used to infect a packaging cell line (along with another virus or plasmid encoding rep and cap). In some embodiments, the packaging cell line is HEK293 cells. The rAAV particles or particles within an rAAV preparation disclosed herein, may be of any AAV serotype, including any derivative or pseudotype (e.g., 1, 2, 3, 4, 5, 6, 7, 8, 9, 10, 2/1, 2/5, 2/8, 2/9, 3/1, 3/5, 3/8, or 3/9).

### Supporting Therapies

In several embodiments, multiple doses of rAAV are administered to a patient over time. In some instances, a patient (or subject) may have an immune response to a second (or subsequent) dose of AAV. In several embodiments, a patient may have had a prior exposure to AAV and thus could have an immune response to a first dose of rAAV. To reduce and/or eliminate such an immune response, In several embodiments, an immunomodulatory regimen (or immunosuppression regimen) is administered to the patient. In several embodiments, the regimen comprises an antibody and/or another immune modulatory agent. The antibody and/or another immune modulatory agent may be administered before, after, or simultaneously with the first dose of rAAV. The antibody and/or another immune modulatory agent may be administered before, after, or simultaneously with the second dose of rAAV. In several embodiments, the antibody comprises an antibody directed against CD20, such as a monoclonal antibody. In several embodiments, a Type I or Type II monoclonal antibody is used. The antibody may be selected from rituximab, ofatumumab, tositumomab, GA101, ibritumomab, ocrelizumab, obinutuzumab, and combinations thereof. In some embodiments, the antibody is Obinutuzumab. Other agents that result in B-cell depletion in the subject may also be used. In several embodiments, an additional agent is administered prior to, in conjunction with, and/or after the monoclonal antibody. In several embodiments, such an agent is an mTOR pathway inhibitor. In several embodiments, the mTOR inhibitor is selected from rapamycin or a rapalog, deforolimus, everolimus, temsirolimus, sirolimus, or combinations thereof. In some embodiments, the immunomodulatory regimen is a combination of obinutuzumab and rapamycin. In several embodiments, an additional agent is administered that functions to either disrupt the TLR9-MyD88-type I interferon (IFN) signaling pathway and/or neutralize Type I IFNs, thereby inhibiting the immune response directed against the viral vector. In several embodiments, the anti-CD20 antibody and the mTOR pathway inhibitor are each administered more than once. In several embodiments, the anti-CD20 antibody and the mTOR pathway inhibitor are each administered more than once and the anti-CD20 antibody and the mTOR pathway inhibitor are each administered to a subject at least once prior to the administration of a second dose of (or administration of the subsequent) rAAV vector and at least once prior to the administration of a first dose of (or administration of the prior) rAAV vector, in accordance with the methods disclosed herein. Effective doses for these agents are readily determined, for example by titrating a dose to reduce B-cell count and/or maintain a desired serum trough level between doses. Additional information on immunomodulation can be found in U.S. patent application Ser. Nos. 12/827,520, 15/306,139, or 16/742,737, the entire contents of each of which is incorporated by reference herein. In various embodiments, the subject or patient is human.

### Administration and Dosing

In certain embodiments, treatment of a subject with vectors and/or supporting therapies as described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. Each of these comparisons are versus, for example, a different therapy for a disease, which includes vectors that do not express the constructs disclosed herein. In some embodiments, the subject is a mammal. In some embodiments, the subject is a primate, such as a non-human primate. In some embodiments, the subject is a human.

Administration can be by a variety of routes, including, without limitation, intravenous (IV), intrathecal (IT), intracerebroventricular, intra-arterial, subcutaneous, intramuscular, intrahepatic, intraperitoneal and/or local delivery (e.g., direct injection and/or perfusion of an organ) to an affected tissue. In several embodiments, combinations of delivery routes are used, either sequentially or concurrently. For example, in several embodiments, intravenous delivery is used in combination with intrathecal delivery to administer one or more types of rAAV. In some embodiments, the rAAV particle or composition are administered via intravenous injection. Some embodiments further include administering the rAAV particle or composition via intrathecal injection or administering the rAAV particle or composition via intracisternal injection. In some embodiments, the rAAV particle or composition are administered via intrathecal injection or administered via intracisternal injection.

Dosages of a viral vector can depend primarily on factors such as the condition being treated, the age, weight and health of the patient, and may thus vary among patients. For example, a therapeutically effective human dosage of the viral vector is generally in the range of from about 0.1 ml to about 100 ml of solution containing concentrations of from about 1×10⁹ to 1×10¹⁶ genomes of viral vector. A preferred human dosage can be about 1×10¹³ to 1×10¹⁶ AAV genomes. In several embodiments, the dose is based on the approximate mass of the target organ, ranging for example from about 1×10¹⁰ viral genomes per kg (vg/kg) of the target organ to about 1×10¹⁶ viral genomes per kilogram of the target organ, including, but not limited to about 1×10¹² vg/kg target organ, about 6×10¹² vg/kg target organ, about 9×10¹² vg/kg target organ, about 1×10¹³ vg/kg target organ, about 6×10¹³ vg/kg target organ, about 9×10¹³ vg/kg target organ, about 1×10¹⁴ vg/kg target organ, about 6×10¹⁴ vg/kg target organ, about 9×10¹⁴ vg/kg target organ, about 1×10¹⁵ vg/kg target organ, about 1.5×10¹⁵ vg/kg target organ, about 3×10¹⁵ vg/kg target organ, about 6×10¹⁵ vg/kg target organ, about 9×10¹⁵ vg/kg target organ, or any dose between those listed. In several embodiments, the target organ is the heart. In several embodiments the target organ is the brain. The dosage will be adjusted to balance the therapeutic benefit against any side effects and such dosages may vary depending upon the therapeutic application for which the recombinant vector is employed. The levels of expression of the transgene can be monitored to determine the frequency of dosage resulting from the vector.

Several embodiments employ multiple routes of administration wherein different (or optionally the same) amount of AAV is administered. In several embodiments, a ratio of AAV dose is used for two routes of administration with the ratio ranging from 1:2, 1:4, 1:6, 1:8, 1:10, 1:15, 1:20, 20:1, 15:1, 10:1, 8:1, 6:1, 4:1, 2:1, or 1:1 (or any ratio between those listed).

Some embodiments relate to a method of treating Friedreich's ataxia. The method comprises: administering a therapeutically effective amount of a recombinant adeno-associated virus (rAAV) particle to a subject having Friedreich's ataxia, wherein the rAAV particle comprises an rAAV vector encoded by a nucleic acid comprising an expression construct, the expression construct comprising: a human frataxin (FXN) coding sequence and a truncated human FXN 3′ untranslated region (UTR), a promoter operably linked to the human FXN coding sequence and truncated human FXN 3′ UTR, wherein the expression construct is flanked on each side by an inverted terminal repeat sequence; and wherein the rAAV particle is administered by intravenous injection, and further rAAV particles are administered by intrathecal or intracisternal injection, wherein the ratio of rAAV particles administered to the subject via intravenous injection to rAAV particles administered to the subject via intrathecal or via intracisternal injection is 1:10.

Some embodiments relate to a method of treating Friedreich's ataxia. The method comprises: administering a therapeutically effective amount of a composition comprising a plurality of rAAV particles to a subject having Friedrich's ataxia, wherein the rAAV particles comprise a nucleic acid comprising rAAV vector comprising an expression construct comprising a human frataxin (FXN) coding sequence and a truncated human FXN 3′ untranslated region (UTR) operably linked to a promoter, wherein the expression construct is flanked on each side by an inverted terminal repeat sequence, wherein the amount of the composition is administered by intravenous injection, and a further amount of the composition is administered by intrathecal or intracisternal injection, wherein the ratio of rAAV particles within the composition administered to the subject via intravenous injection to the rAAV particles within the composition administered to the subject via intrathecal or via intracisternal injection is 1:10.

Additionally, in several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

### Examples

The materials and methods disclosed herein are non-limiting examples that are employed according to certain embodiments disclosed herein.

### Functional Benefit of Frataxin

As discussed above, the goal of gene therapies is to augment, supplement, replace the function of a protein that is malfunctioning, or absent. The following experiments were to determine if delivery of frataxin could in fact lead to functional improvements in a mouse model of Friedreich's ataxia.

Frataxin is a mitochondrial iron-binding protein works in mitochondria to regulate iron and sulfur molecules, while also working as an antioxidant to reduce oxidative stress. It functionally aids mitochondria in efficiently processing ATP, the source of cellular energy. Decreased frataxin levels in FA lead to the inability of iron-sulfur clusters to form in mitochondria, causing a reduction of energy production, and additionally make mitochondria sensitive to free radicals. Cells in the brain, spinal cord, and muscles become damaged and degenerate, causing signs and symptoms of FA. As discussed above, FA is a rare, inherited, progressive disease affecting the nerves and spinal cord, causing loss of control of body movements (ataxia). FA is a multi-system disease with cardiac effects (hypertrophic cardiomyopathy, progressive and severe, and early morbidity and mortality, clinically significant arrhythmia), skeletal abnormalities (scoliosis, pes cavus), hearing loss (auditory neuropathy/dyssynchrony, vestibular dysfunction) endocrine irregularities (diabetes rates between 8-32% and glucose intolerance in ˜49%), ocular symptoms (nystagmus, oculomotor disturbances), and general fatigue. Mortality in FA is most commonly due to cardiac complications (accounting for nearly 60% of deaths). FA is an autosomal recessive triplet repeat disorder. Normally, there are between about 6 and 36 GAA repeats between exon 1 and exon 2 of frataxin. However, in FA, this GAA repeat can occur between about 70 to 1700 times, which can relate to severity of disease. Given that FA is an autosomal recessive disorder (each parent being an unaffected carrier), there is a 1:4 chance of the non-functional gene being passed from both parents to a child, regardless of the child's gender.

Advantageously, as disclosed herein in certain embodiments, gene therapy for FA can be beneficial due to the monogenic nature of the disease, with the phenotype of the disease being directly correlated to the amount of frataxin. All patients, express some frataxin and no aberrant protein, so the immune system will recognize frataxin as a self-protein. Moreover, because of the multiple systems affected by FA, targeting one or more affected organs will be clinically meaningful.

FIG. 1 shows a plasmid map of a non-limiting embodiment of a viral construct according to embodiments disclosed herein. In several embodiments, the transgene of interest comprises a codon-optimized human frataxin gene. In several embodiments, this is followed by a 3′-UTR (untranslated region). In several embodiments, the 3′-UTR functions to confer post-transcriptional control on the expression of frataxin via the endogenous regulatory elements. In several embodiments, the encoded frataxin undergoes maturation within the mitochondria of the host organism having received the rAAV vector.

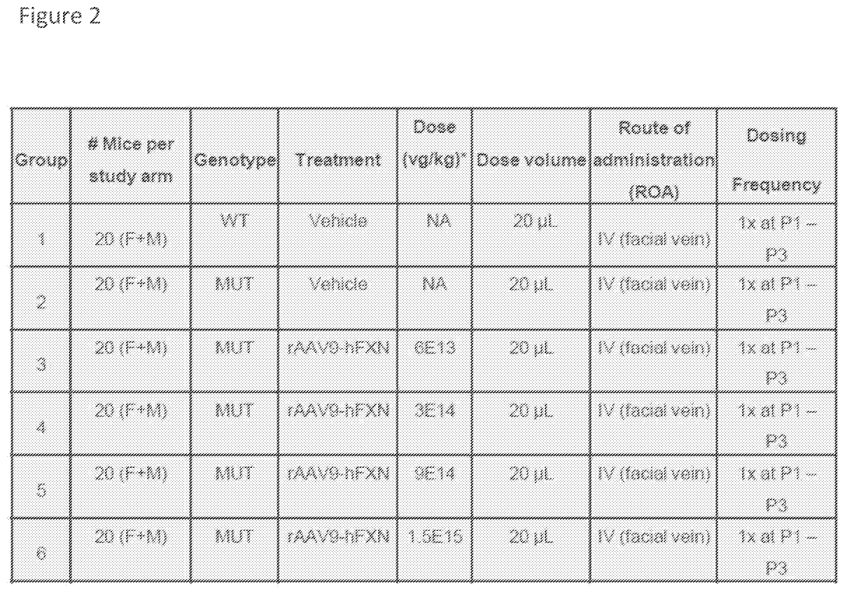

FIG. 2 shows a matrix for the experimental design to confirm that administration of a viral vector for generating frataxin imparts functional benefit. The mice used in these groups were either wild-type (positive control), mutant mice receiving vehicle (negative control), or mutant mice receiving one of four doses of rAAV9-hFXN. Mice were Fxn^{flox/null}::MCK-Cre (also known B6.Cg-Fxn^{em2Lutzy} Fxn^{em2.1Lutzy} Tg(Ckmm-cre)5Khn/J from the Jackson Laboratory), which have a Cre-conditional frataxin allele, a global knockout frataxin allele and a cardiac/skeletal muscle-specific Cre recombinase transgene. Mice were dosed as indicated.

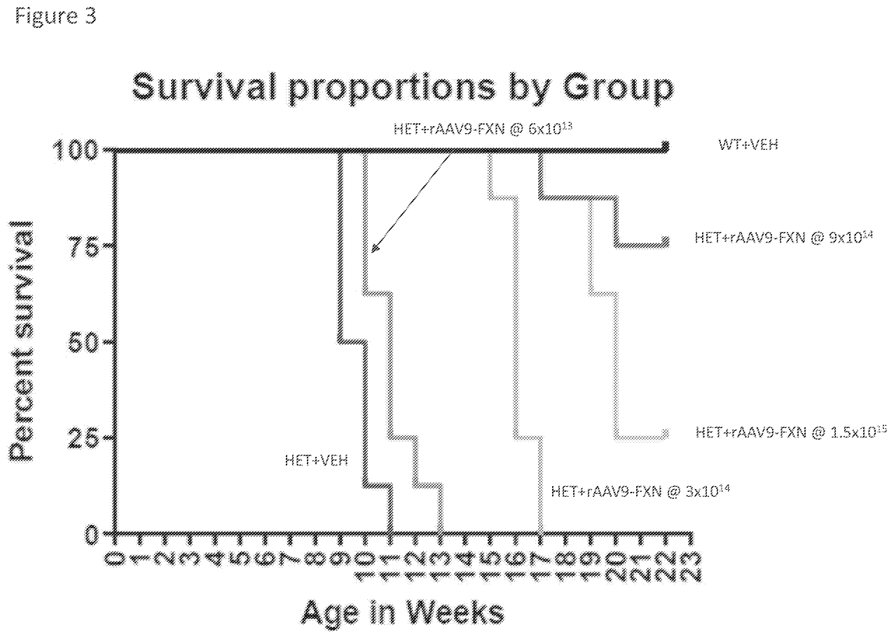

FIG. 3 shows survival curves for the test groups receiving rAAV comprising “FXN01” (SEQ ID NO: 11). As expected, wild-type mice all survived the 23 weeks of the experiment. Mutant negative control mice survived through 11 weeks. Each of the groups that received rAAV9-hFXN01 had improved survival, with the three highest doses exhibiting over 75% survival through 15 weeks and the two highest doses exhibiting over 75% survival for 19 weeks. One group test group maintained 75% survival throughout the duration of the experiment. These data demonstrate that, at a fundamental assessment of survival, administration of rAAV vectors encoding frataxin leads to improved survival outcomes.

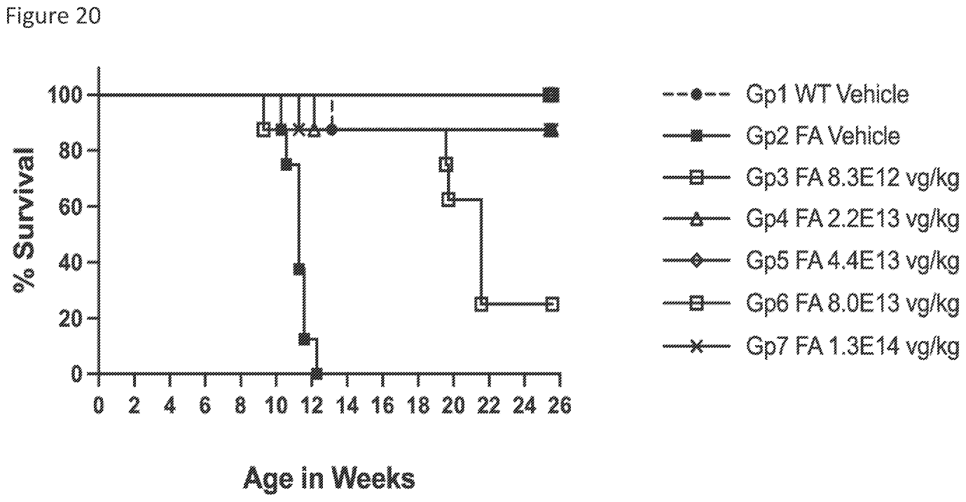

FIG. 20 shows survival curves for test groups receiving rAAV comprising the improved codon-optimized FXN sequence (SEQ ID NO: 2). Animals at postnatal days 2 and 3 (p2-p3) received a single dose in 20 microliter volumes as indicated in FIG. 20 through intravenous administration via the superficial temporal vein. As shown in FIG. 20, the negative control mutant mice receiving only vehicle (“Gp2 FA vehicle”) did not survive past ˜12 weeks. However, each treatment group receiving rAAV comprising SEQ ID NO: 2 had animals surviving out to 26 weeks, with all but the lowest dose group having greater than 80% survival at 26 weeks (FIG. 20). These data demonstrate that, at a fundamental assessment of survival, administration of rAAV vectors encoding an improved codon-optimized frataxin sequence leads to improved survival outcomes.

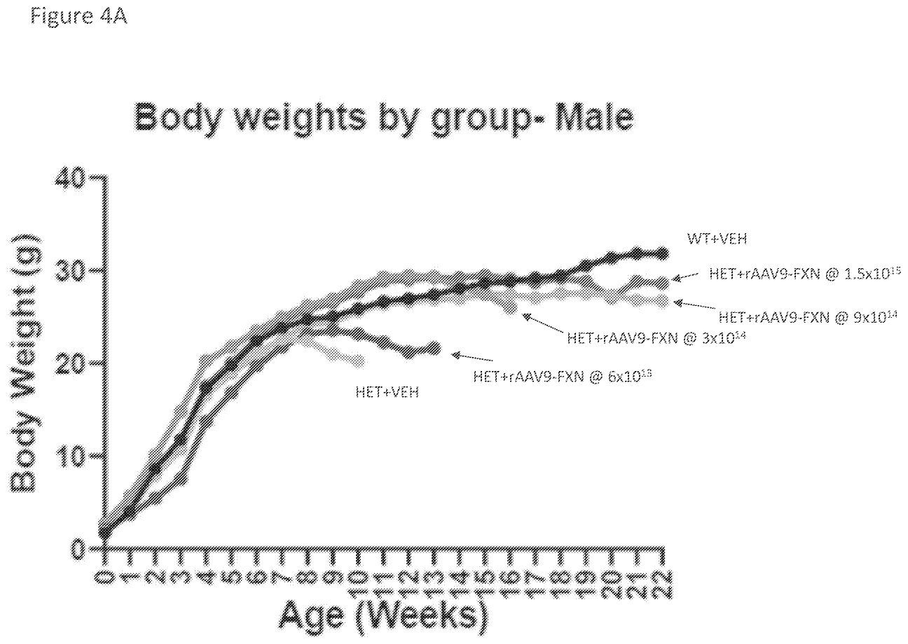

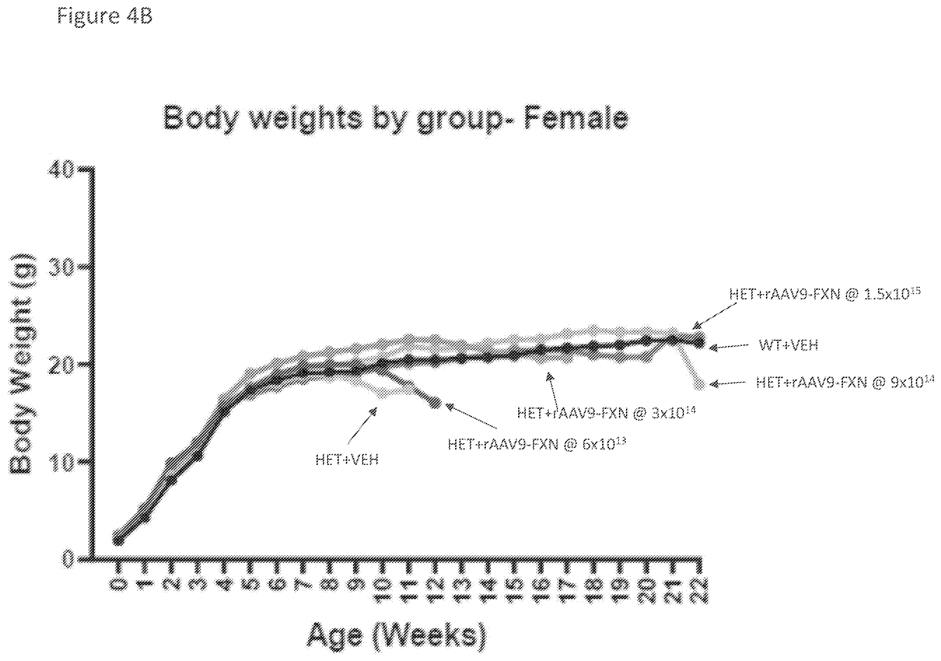

Given the inefficient processing of ATP in mitochondria of FA patients, body weight will often decline. FIGS. 4A and 4B show longitudinal tracking of body weight in male (4A) and female (4B) mice in the indicated treatment groups. Both genders of mice receiving the higher doses of rAAV encoding frataxin exhibited improved maintenance of body week until at least week 20 of the experiment. According to several embodiments, administration of an AAV vector encoding frataxin leads to improved maintenance of body weight and/or muscle mass.

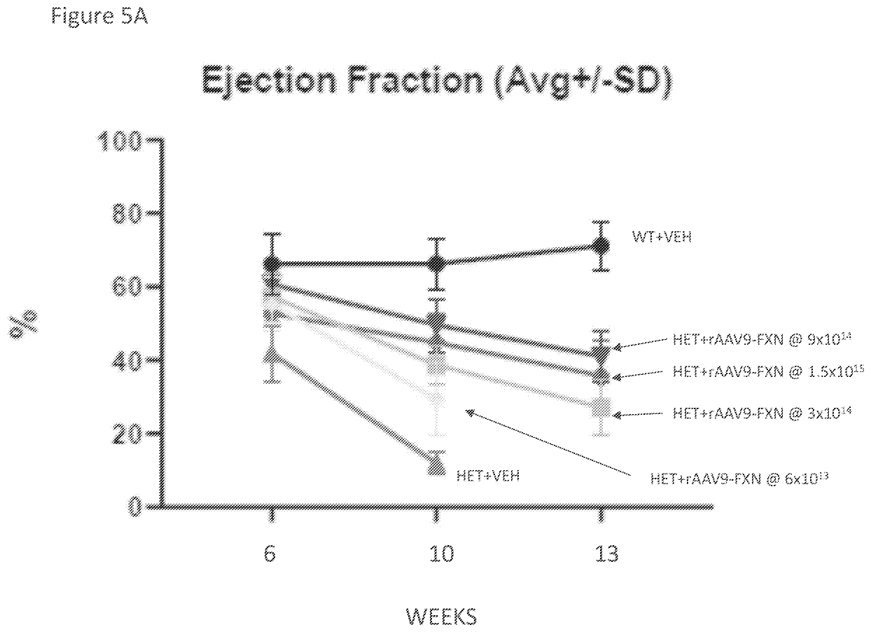

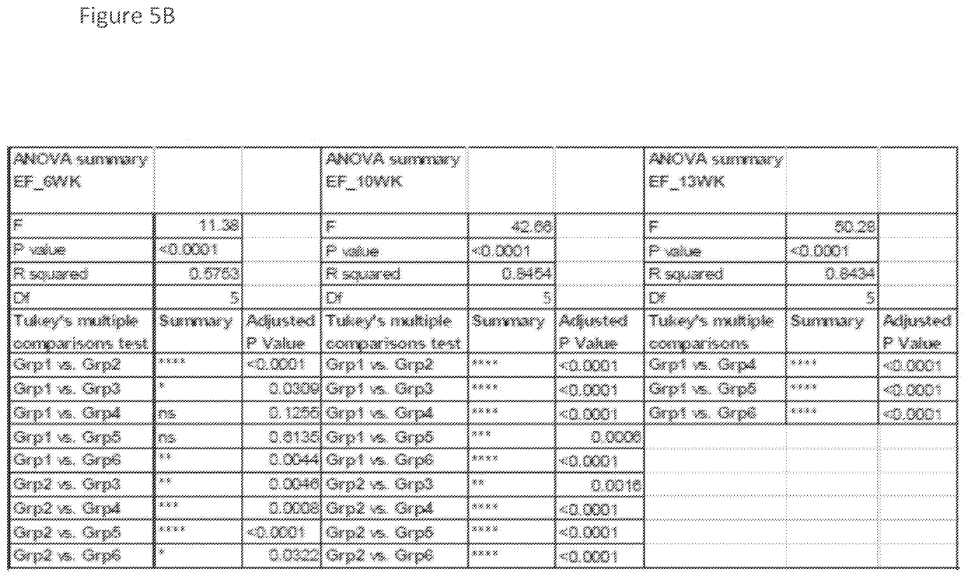

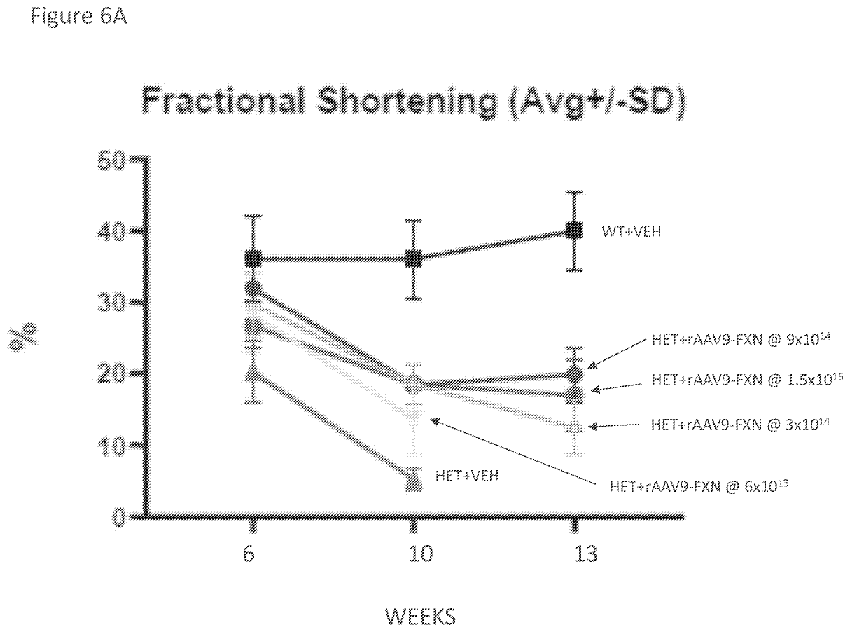

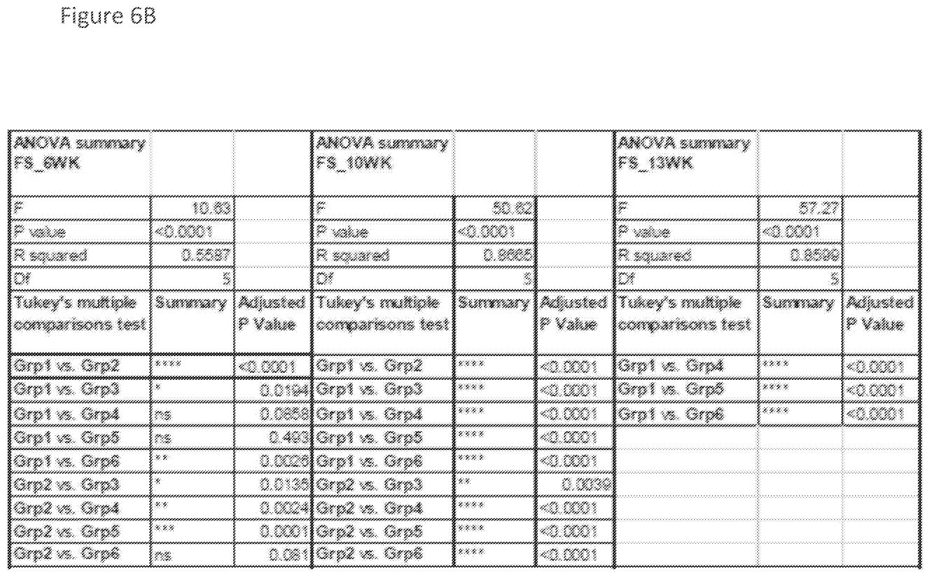

As discussed above, cardiac complications account for a significant amount of the mortality of FA patients. In-life cardiac function measurements were conducted to assess the functional benefit associated with frataxin expression. Cardiac function may be measured by ejection fraction, or any other method known in the art. Measurements were taken 6, 10, and 13 weeks after rAAV-hFXN01 dosing. FIG. 5A shows ejection fraction data for each group measured at 6, 10 and 13 weeks. FIG. 5B shows the associated statistical analysis. At each of the 6 and 10 week time points, each of the treatment groups exhibited significantly greater ejection fraction than the negative control group. By 13 weeks, ejection fraction had declined in each treatment group. FIG. 6A shows data for fractional shortening of cardiac myocytes (a measure of cardiac function) at each of 6, 10 and 13 weeks. FIG. 6B shows the associated statistical analysis. At 6 weeks, all but the lowest treatment group showed significantly greater fractional shortening. At 10 weeks, all the treatment groups exhibited greater fractional shortening compared to the negative control. By 13 weeks, the next lowest dose treatment group exhibited some decline in fractional shortening, while the two highest dose treatment group maintained relatively consistent fractional shortening was compared to the prior measurement. These data demonstrate significant therapeutic efficacy at maintaining cardiac function due to rAAV-based delivery of frataxin, as is done according to several embodiments disclosed herein. In several embodiments, multiple doses of rAAV-hFXN reduce or eliminate declining cardiac function.

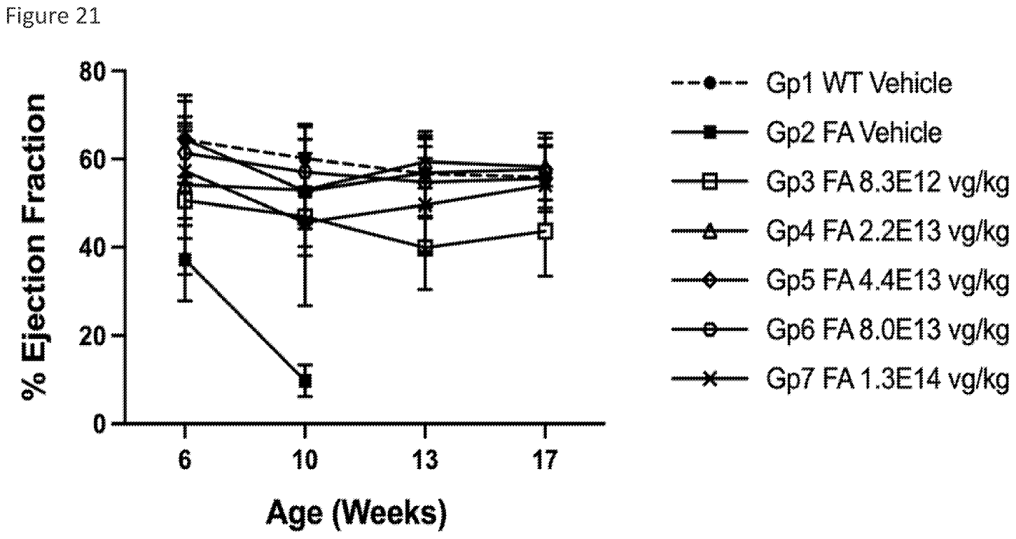

Ejection fraction was also evaluated in animals receiving rAAV comprising the improved codon-optimized frataxin sequence (SEQ ID NO: 2). As shown in FIG. 21, at 10 weeks the negative control mutant mice group (“Gp2 FA vehicle”) had significantly reduced ejection fraction than the positive control wildtype mice and treatment groups and did not survive past ˜12 weeks. However each of the treatment groups, including the lowest dose group, had ejection fraction levels that were maintained at greater than ˜40% through 17 weeks, with certain treatment groups having levels that were not statistically different than the wildtype group levels.

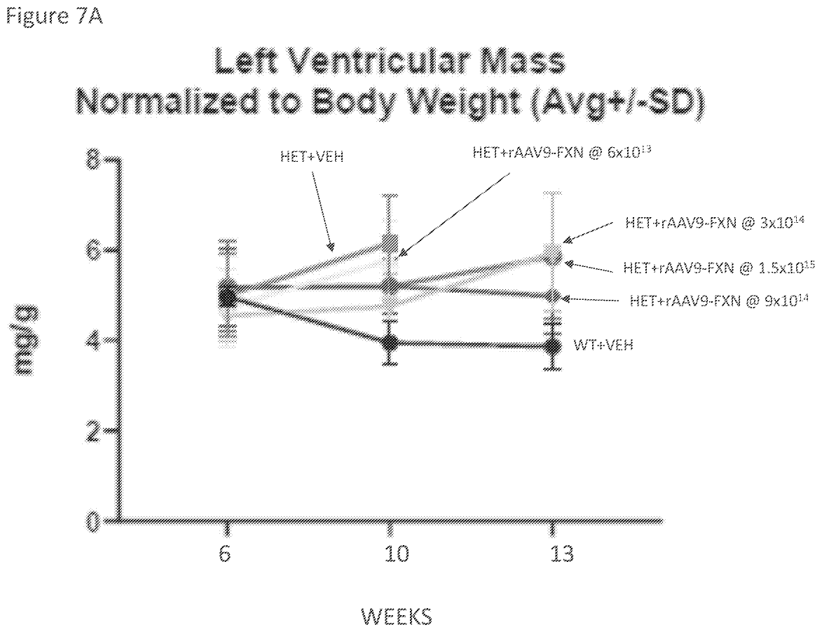

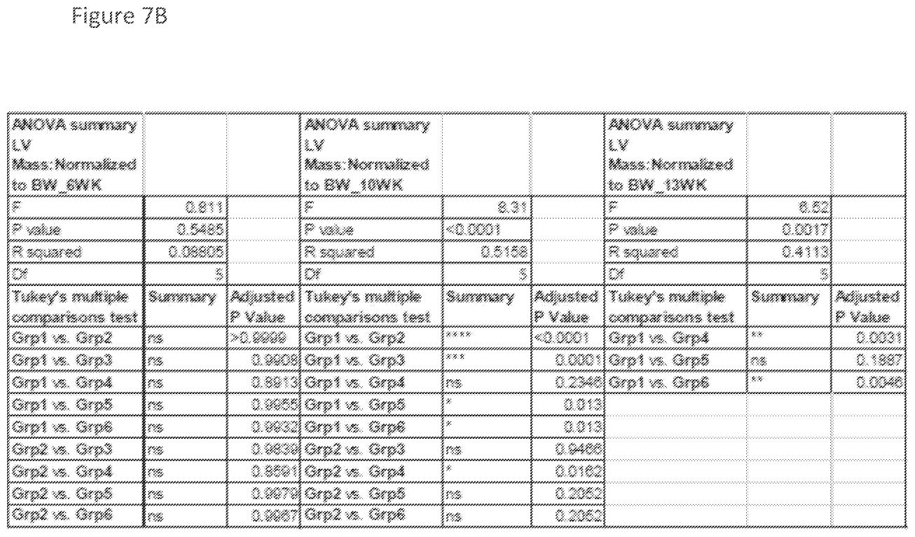

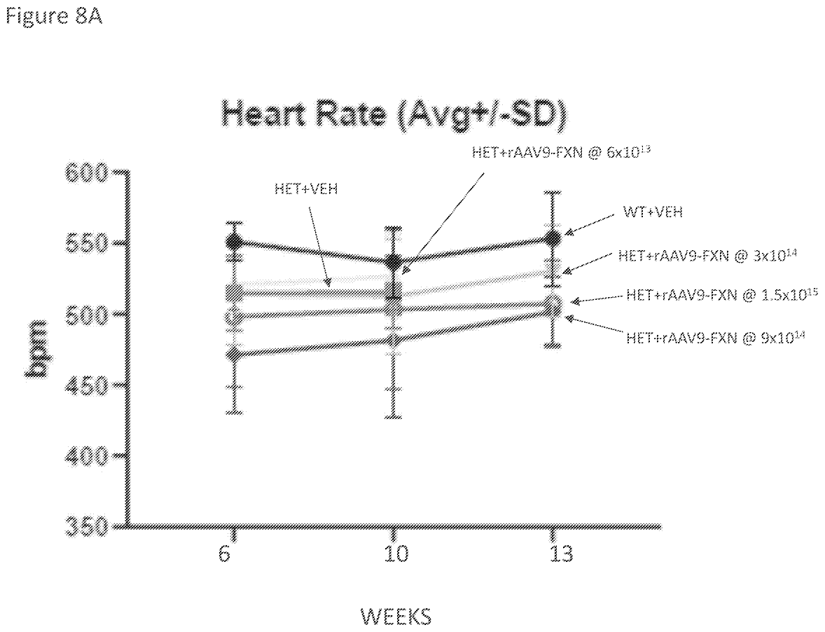

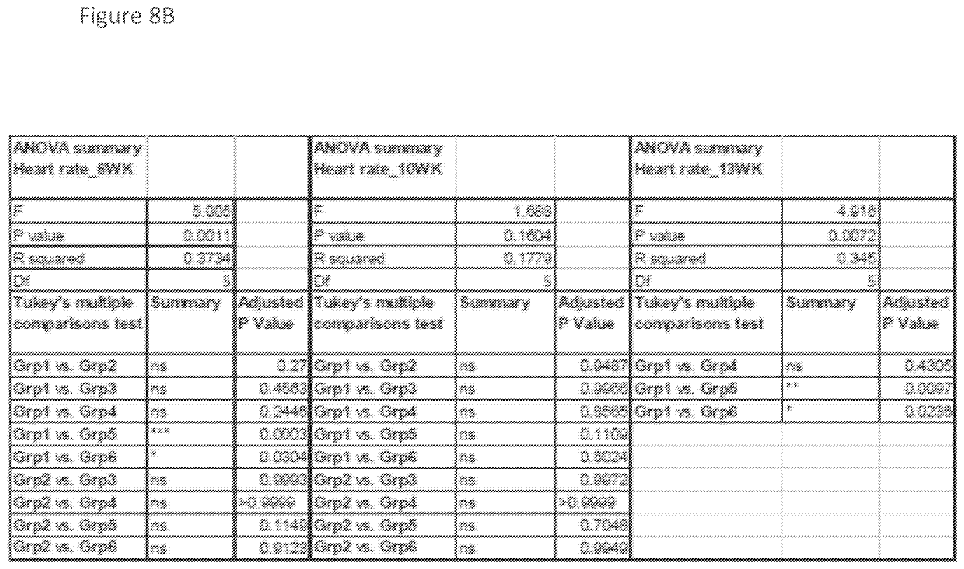

FIG. 7A shows data for left ventricular mass normalized to body weight for each group measured at 6, 10 and 13 weeks. FIG. 7B shows the associated statistical analysis. No differences in left ventricular mass were detected at 6 weeks. By 10 weeks, left ventricular mass had increased in one of the treatment groups (lowest dose). There was some increase in left ventricular mass by 13 weeks in two treatment groups, with one group not being significantly different from control wild-type mice. FIG. 8A shows data for heart rate at 6, 10 and 13 weeks. FIG. 8B shows related statistics. As can be seen, heart rate was relatively consistent among the time points. The second lowest dose treatment group did increase heart rate enough to not be significantly different from wild-type control, and both the two highest dosed groups trended in that direction as well. In several embodiments, administration of rAAV-hFXN can beneficially maintain cardiac function, as measured by heart rate.

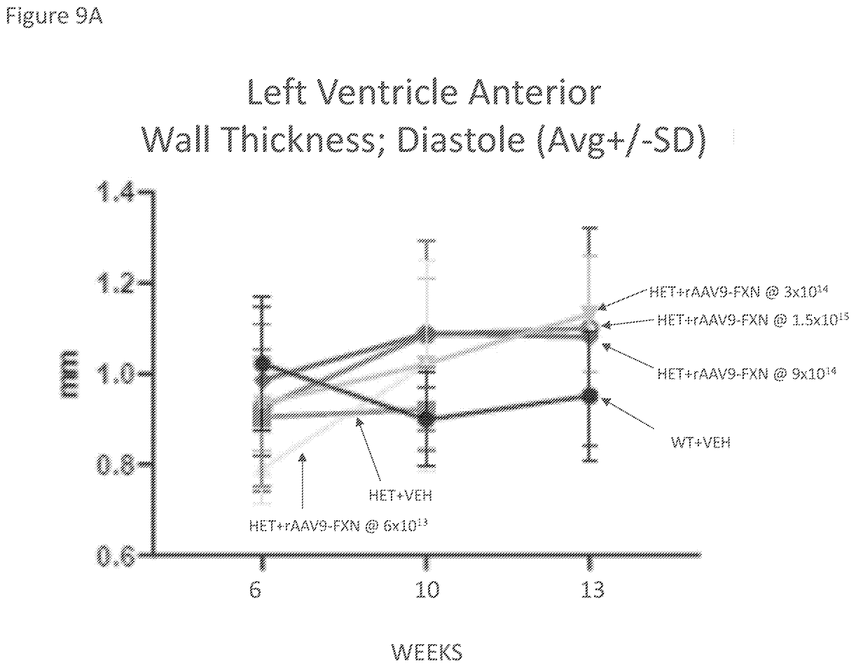

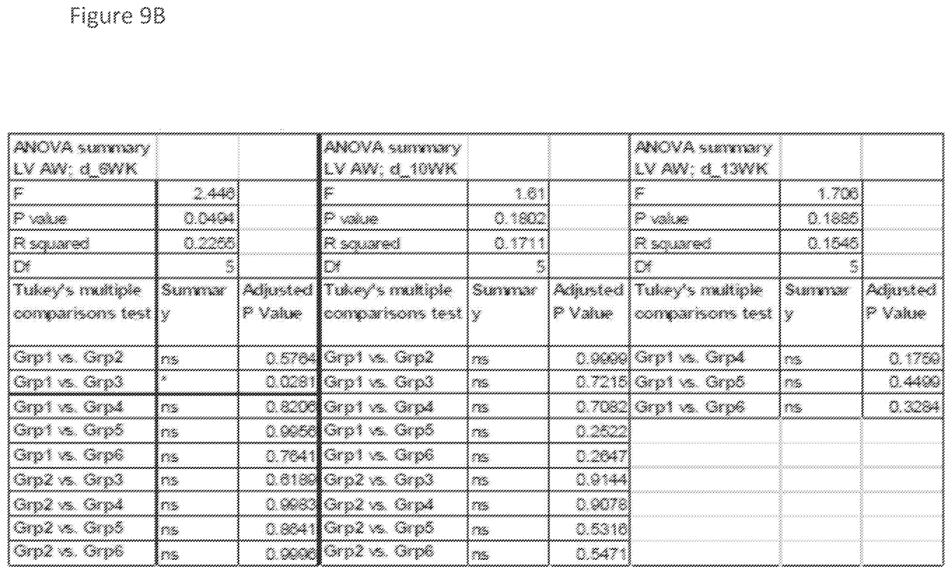

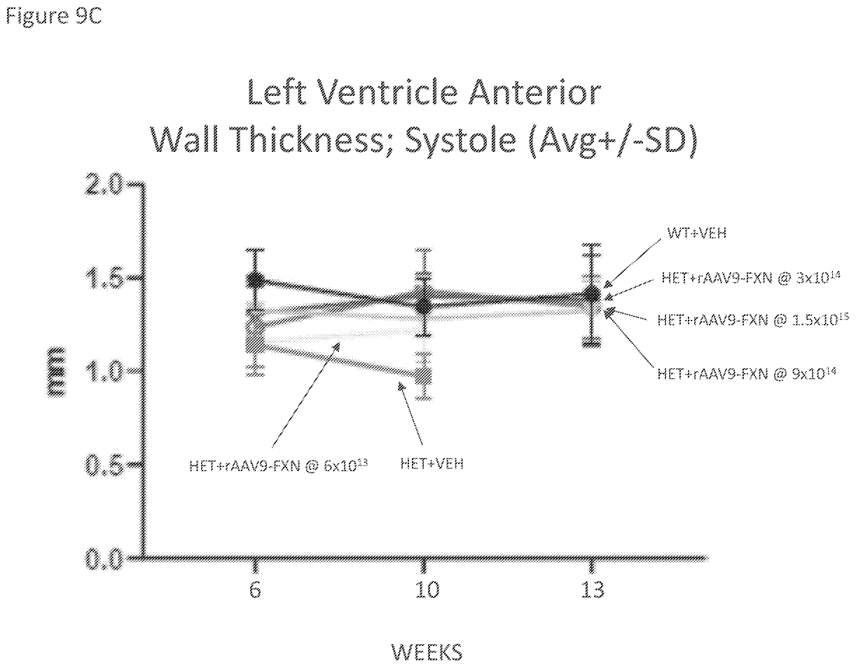

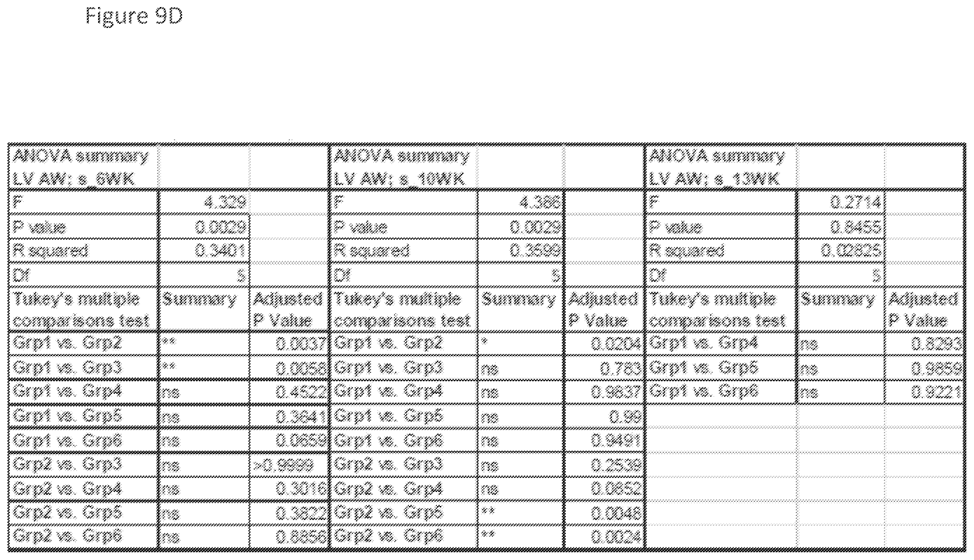

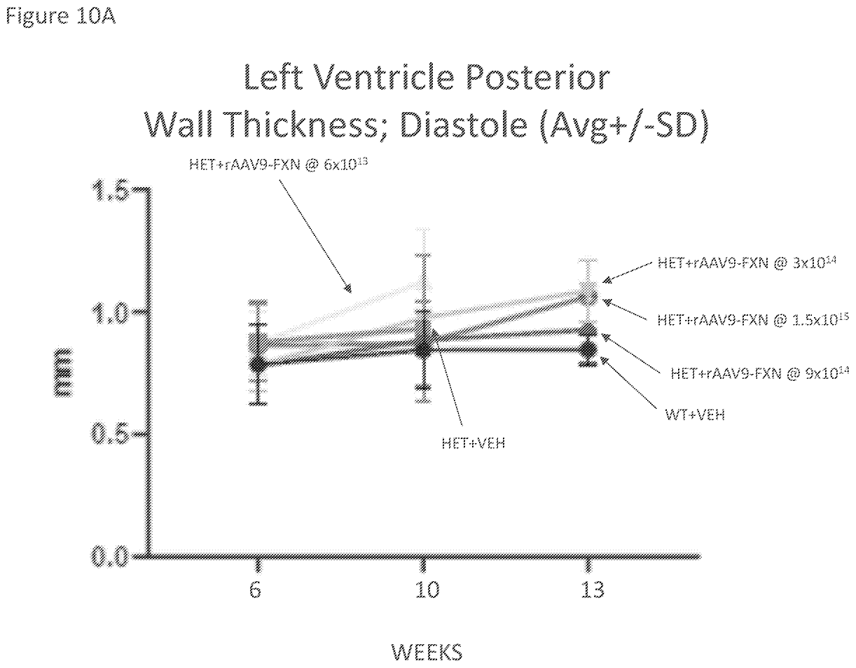

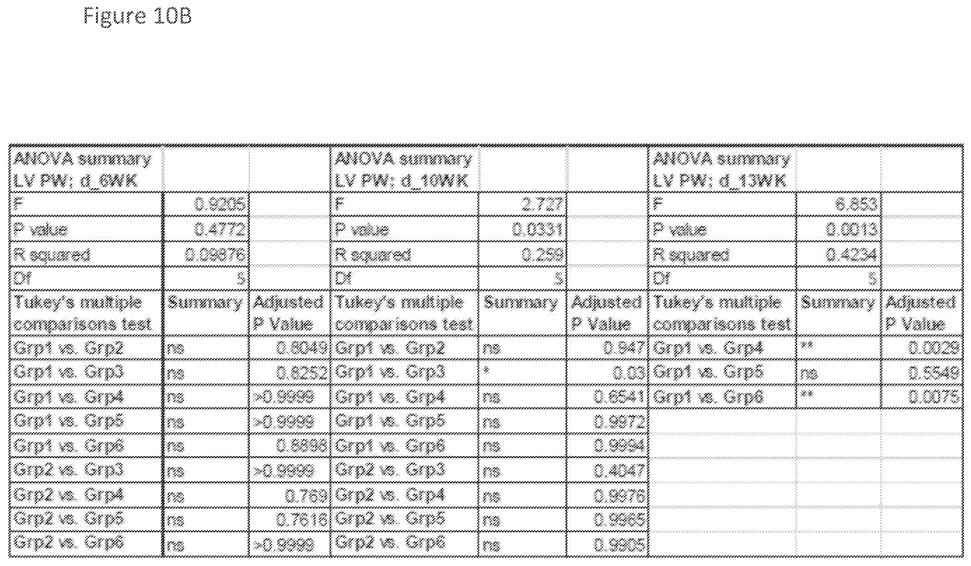

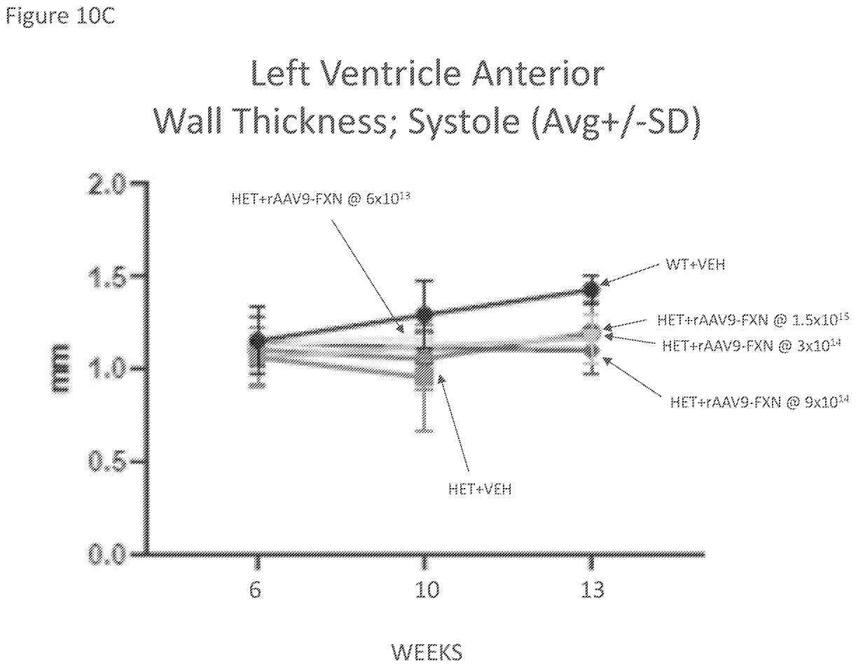

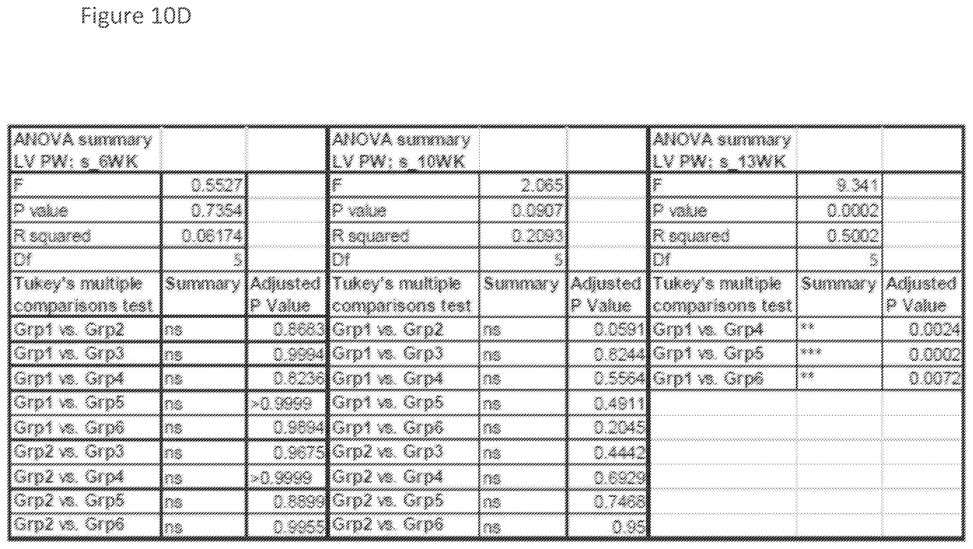

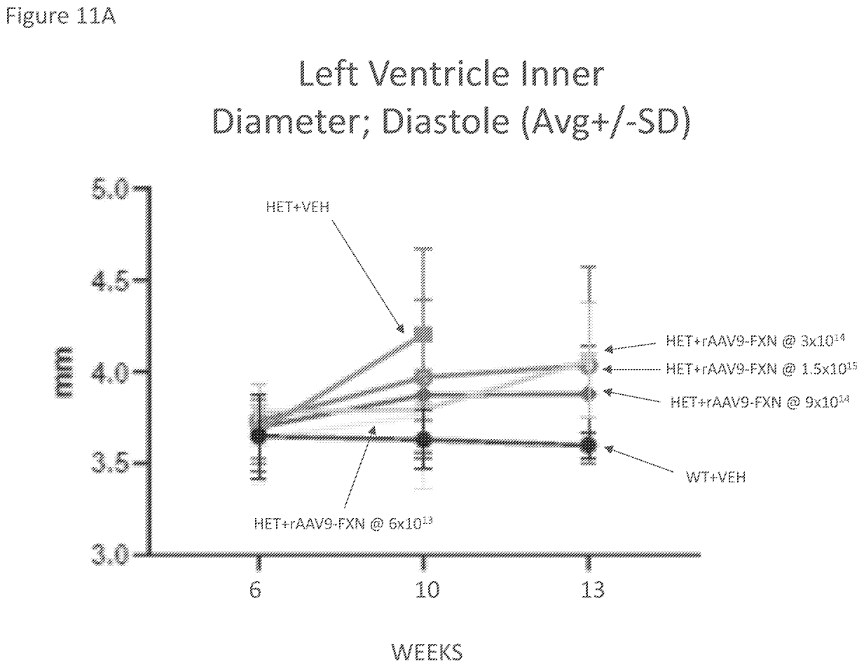

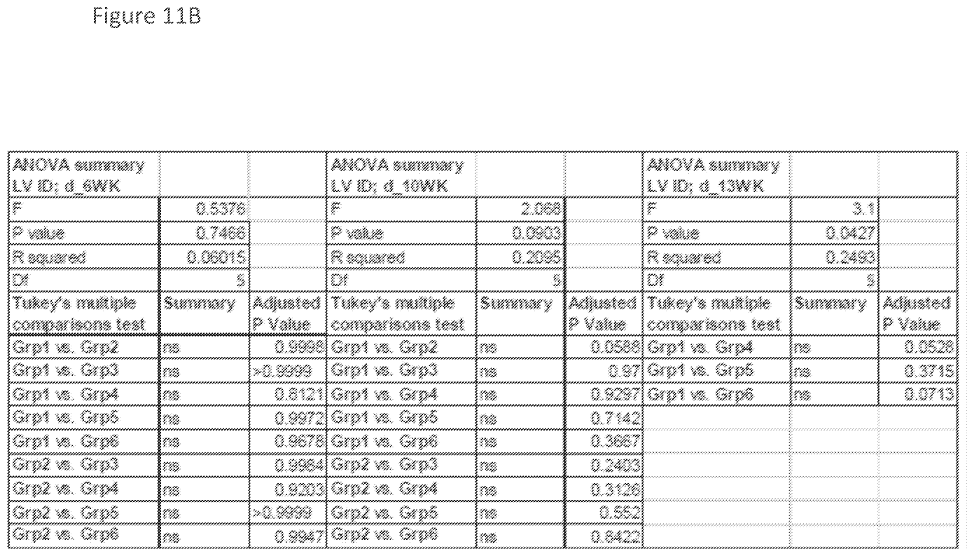

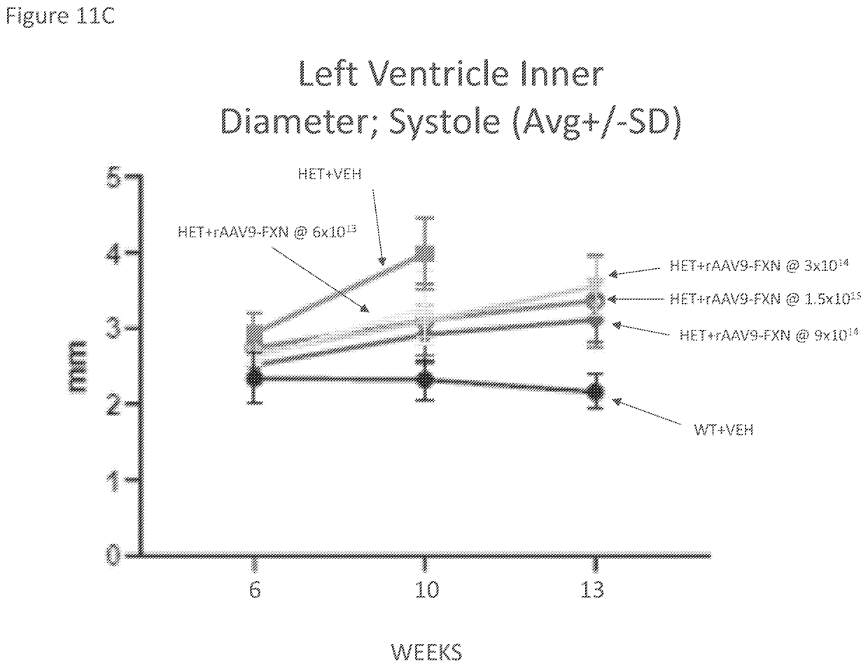

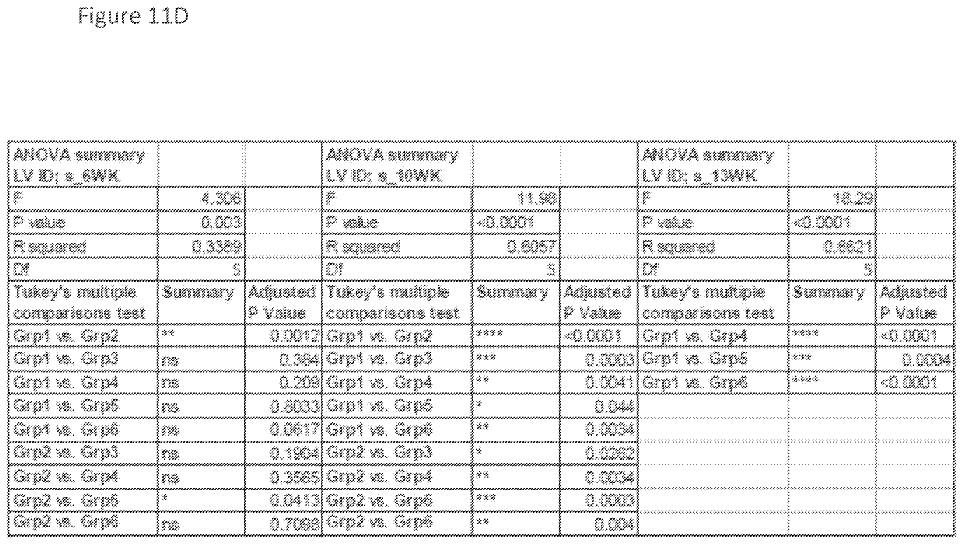

FIGS. 9A and 9C show data related to the thickness of the anterior wall of the left ventricle during diastole (9A) and systole (9C). FIGS. 9B and 9D show corresponding statistics. But for the lowest treatment dose at 6 weeks, none of the treatment groups varied significantly from wild-type control wall thickness during diastole. Similarly, during systole, this treatment group was different from wild-type control at 6 weeks, but not at 10 weeks. The two higher treatment doses were significantly different from untreated controls at 10 weeks, and not significantly different from wild-type controls at 13 weeks. FIGS. 10A and 10C show data related to related to the thickness of the posterior wall of the left ventricle during diastole (10A) and systole (10C). FIGS. 10B and 10D show corresponding statistics. During diastole, other than the lowest treatment dose at week 10, none of the treatment groups differed from wild-type control at 6 or 10 weeks. By 13 weeks, modest increases in wall thickness were detected in two treatment groups. Similarly, no differences were detected at weeks 6 or 10 during systole. An upwards trend in thickness in the wild-type control groups resulted in differences between that group and the treatment groups at week 13, though the general trend of the treatment groups was also upward. FIGS. 11A and 110 show data related to related to the left ventricular inner diameter during diastole (11A) and systole (11C). FIGS. 11B and 11D show corresponding statistics. No differences were detected among any treatment groups during diastole. During systole, there were significant differences between the positive and negative controls at 6 weeks. Ventricular diameter trended upwards in each treatment groups at 10 and 13 weeks, while the metric in wild-type group decreased, leading to significant differences in this measure. In several embodiments, reductions in the increase of ventricular diameter are accomplished with a repeat dose, or repeat doses, of rAAV-hFXN.

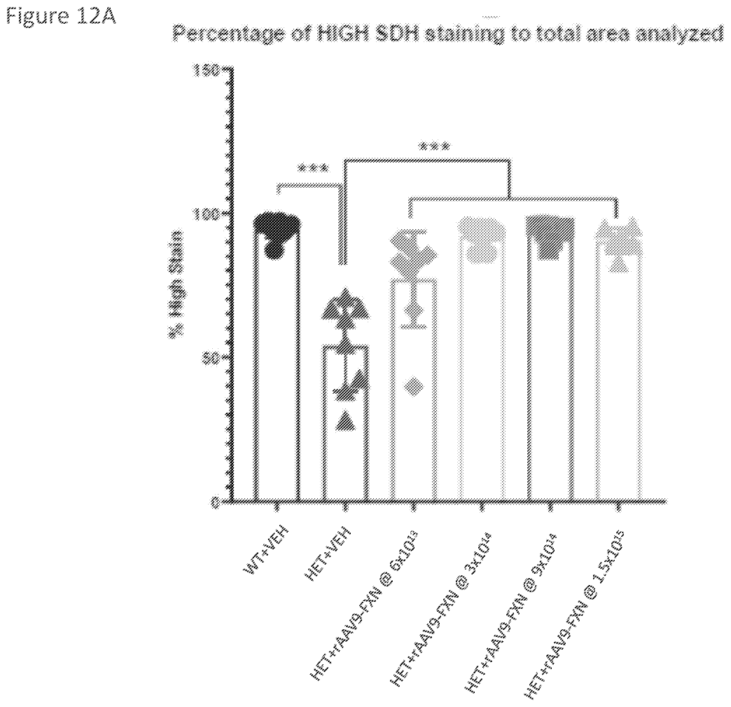

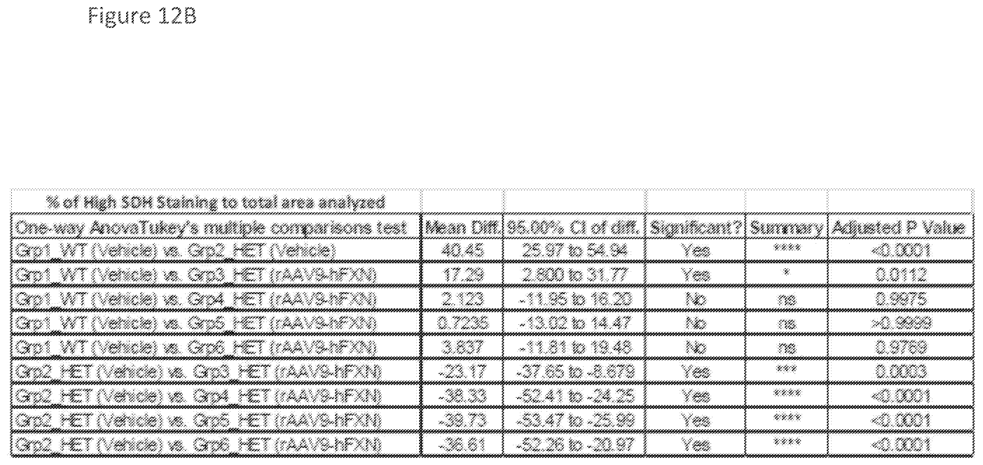

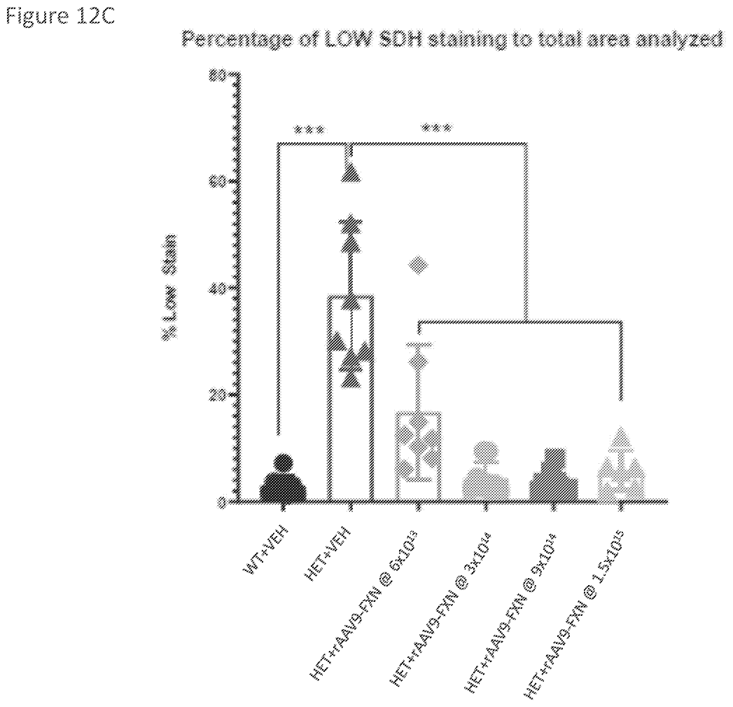

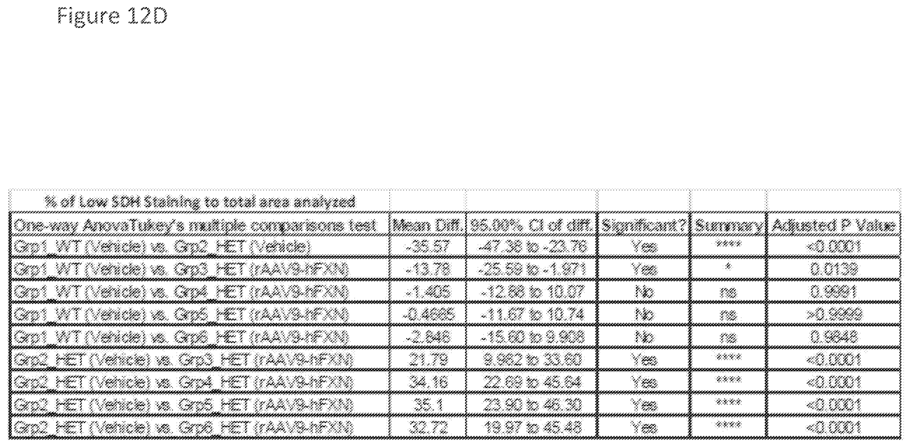

Succinate dehydrogenase (SDH) is an enzyme that bridges the Citric Acid Cycle and the Electron Transport Chain of mitochondria and catalyzes oxidation of succinate to fumarate with the reduction of ubiquinone to ubiquinol. Assessment on SDH staining (e.g., activity) was assessed for each of the groups of mice. Histochemical analysis showing high succinate dehydrogenase in muscle demonstrates high mitochondrial content and high oxidative potential. FIG. 12A shows a histogram summarizing the percentage of high SDH staining detection as compared to the total area of tissue analyzed. FIG. 12B shows the related statistics. As anticipated, wild-type mice showed nearly 100% high SDH staining (12A) and nearly half as much SDH was detected in the mutant untreated group (statistically significant with p-value<0.0001). Regardless of the dose administered each of the treatment groups show SDH staining that was significantly more than the negative control group and only the lowest dose group failed to be not statistically different from the untreated wild-type SDH values. FIG. 12C (and statistics in 12D) confirm these findings from the opposite viewpoint (low SDH staining to total area analyzed). Wild-type and all treatment groups had significantly less low SDH staining and compared to the untreated mutant group (and only the lowest dose treatment group was significantly different from wild-type). These data show that rAAV-hFXN effectively restores SDH activity to nearly wild-type or equivalent to wild-type levels. Taken together, these data show that administration of rAAV-hFXN can support maintained and/or improved cardiac function in FA subjects (e.g., as manifested by improved ejection fraction) and thus viral vectors expressing frataxin can provide functional benefit.

### Expression of Frataxin

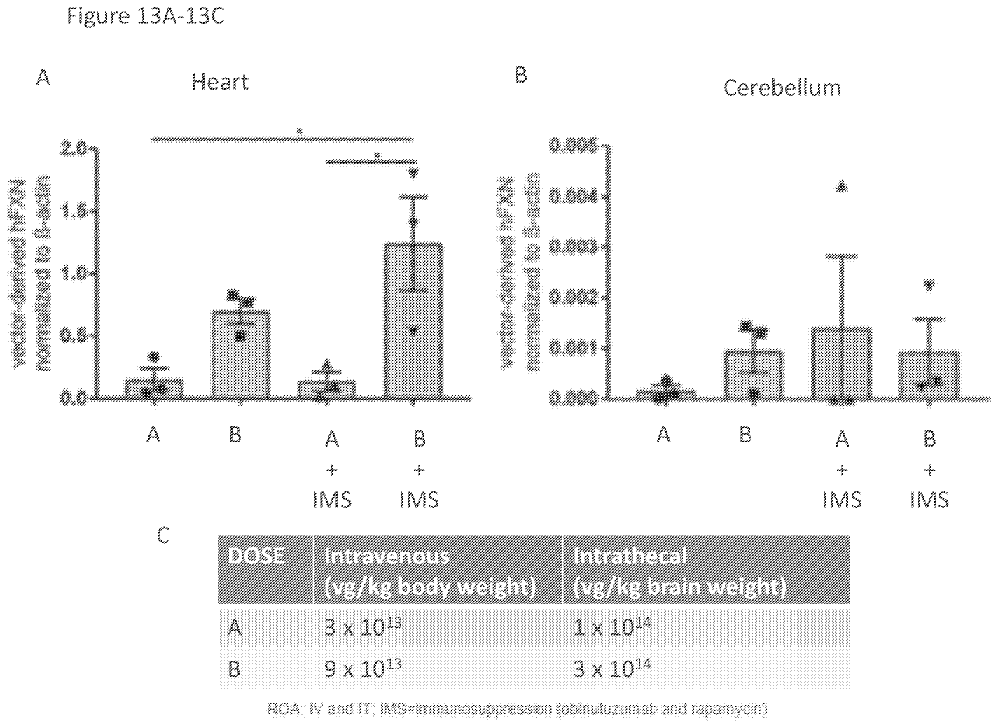

Studies were next undertaken in order to evaluate the expression of frataxin after administration of an rAAV-hFXN construct. This example employed an rAAV9-hFXN as a non-limiting example of a construct suitable for FA gene therapy. FIG. 13A shows detection of human FXN mRNA in the heart of non-human primates 90-days after delivery of rAAV9-hFXN. FIG. 13B shows expression of human FXN in the cerebellum. FIG. 13C shows information on the doses delivered. By way of non-limiting embodiment, the delivery route here was a combination of intravenous and intrathecal administration. These data show dose dependent increases in human FXN mRNA detected in the heart (compare A to B in 13A and 13B). Particularly in the heart tissue, these data also demonstrate that administration of an immunosuppressive regimen can result in significant increases in mRNA of the transgene (here human FXN) when higher doses are administered. By way of non-limiting embodiment, the immunosuppressive regimen here was a combination of obinutuzumab (an anti-CD20 monoclonal antibody) and rapamycin (a mammalian target of rapamycin (mTOR) inhibitor). As discussed above, other immunosuppressive regimens may be used. Similar results are seen in the cerebellum, though the immunosuppressive data is less clear, perhaps due to an impact of the blood brain barrier on the immunosuppressive regimen. Regardless, these data show that the transgene is expressed in a durable fashion, over three months past administration, particularly when elevated doses are administered.

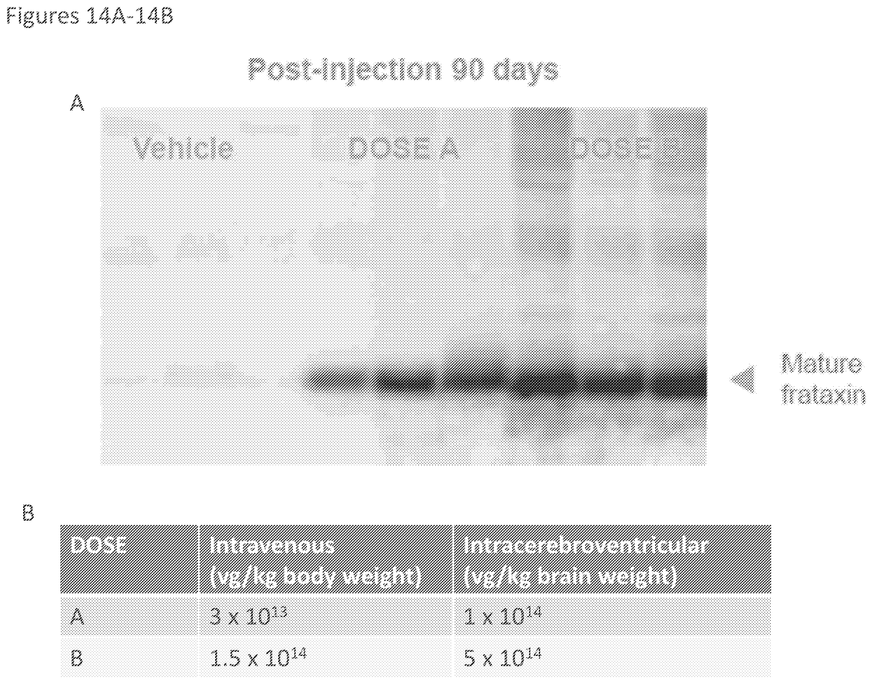

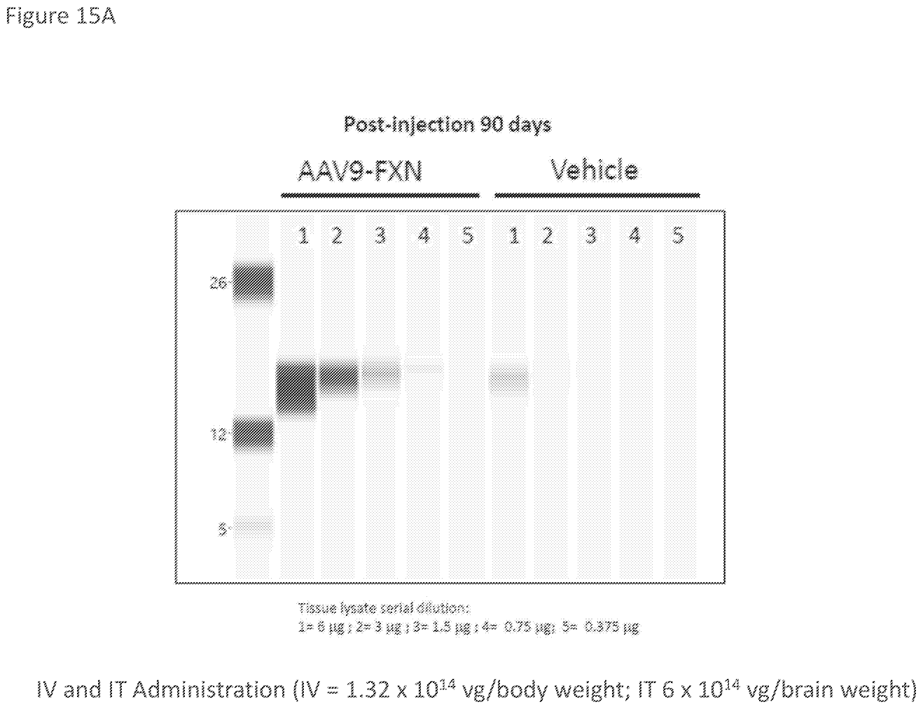

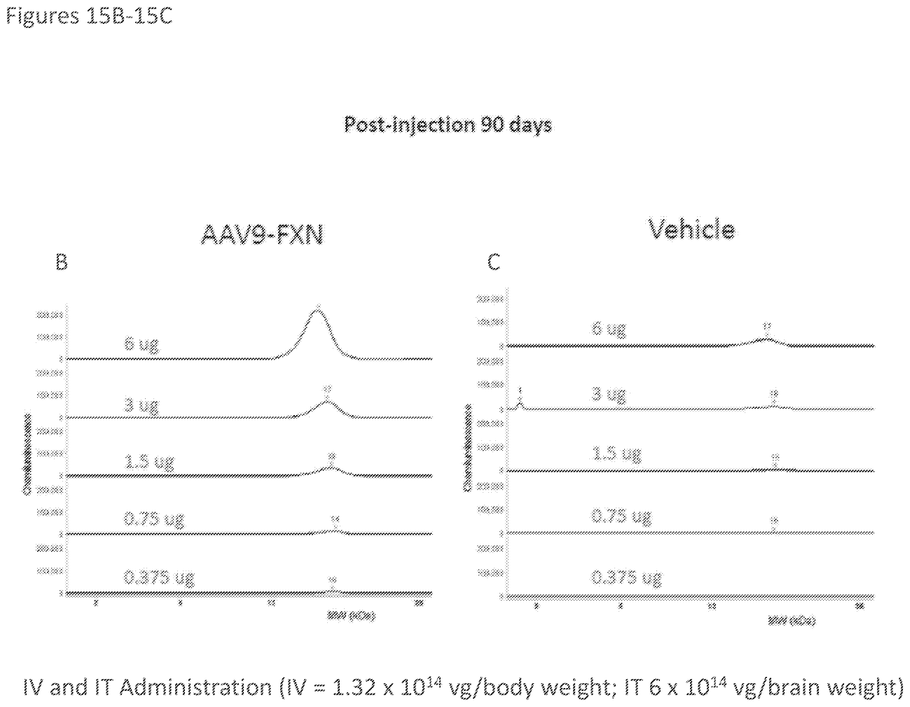

Further evaluating the expression, western blots were performed to detect protein expression. FIG. 14A shows the protein expression data in cardiac tissue of non-human primate after one of the two doses shown in FIG. 14B. Administration here was intravenous and intracerebroventricular, respectively. As can be seen in FIG. 14A, human frataxin protein expression was seen in a dose dependent manner, even 90 days after administration, indicative of durable expression of the human transgene. Further evidencing this, following intravenous and intrathecal administrations of the rAAV vector, human frataxin protein content of cardiac tissue lysates were assessed, shown in FIG. 15A. Chemiluminescence data for treated (FIG. 15B) and vehicle (FIG. 15C) show peaks of signal at the expected molecular weight, indicated that the non-human primate properly process frataxin into the mature human protein.

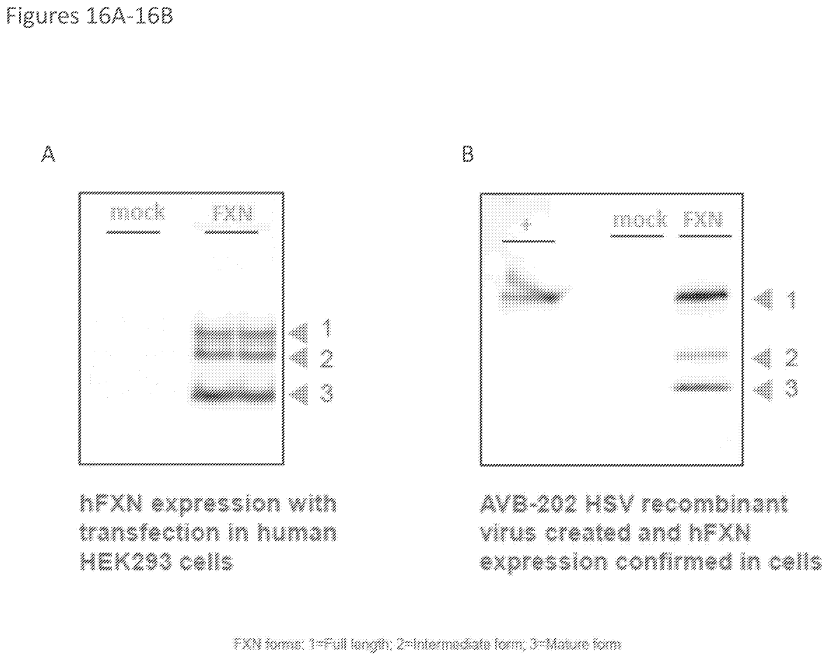

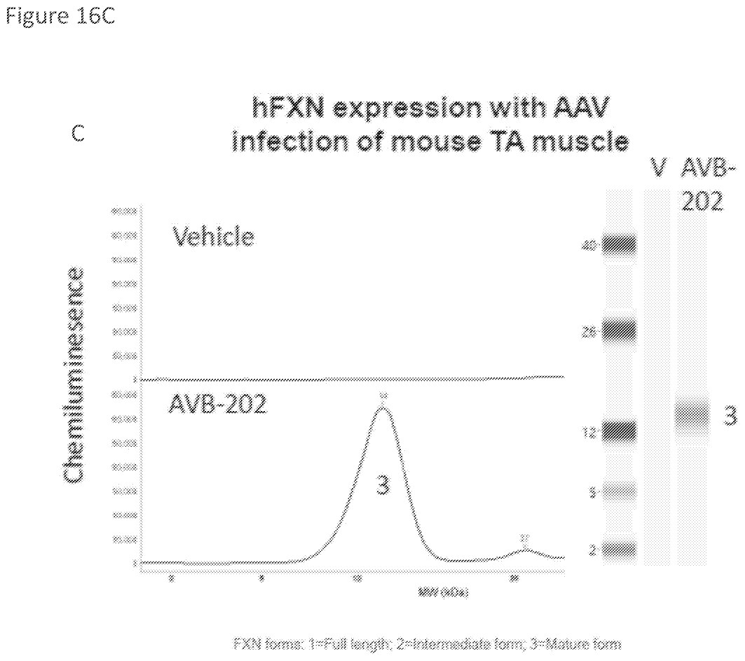

Additional expression data is shown in FIGS. 16A-16C. These specific data result from the use of the codon-optimized frataxin gene disclosed herein (SEQ ID NO: 1, SEQ ID NO: 2). FIG. 16A shows expression of each of the expected frataxin forms (full length, intermediate and mature) when HEK293 cells are transfected with an expression plasmid encoding codon-optimized human frataxin. FIG. 16B shows that expression is maintained when an rAAV9-hFXN vector is generated (using herpes simplex virus-based production) and used to infect HEK293 cells. FIGS. 16C and 16D both demonstrate that mature human frataxin is expressed after viral infection of mice, and subsequent detection of human frataxin in protein lysates from the tibialis anterior. FIG. 16C shows detection of a chemiluminescent signal at the anticipated size of mature frataxin and FIG. 16D shows corresponding protein detection.

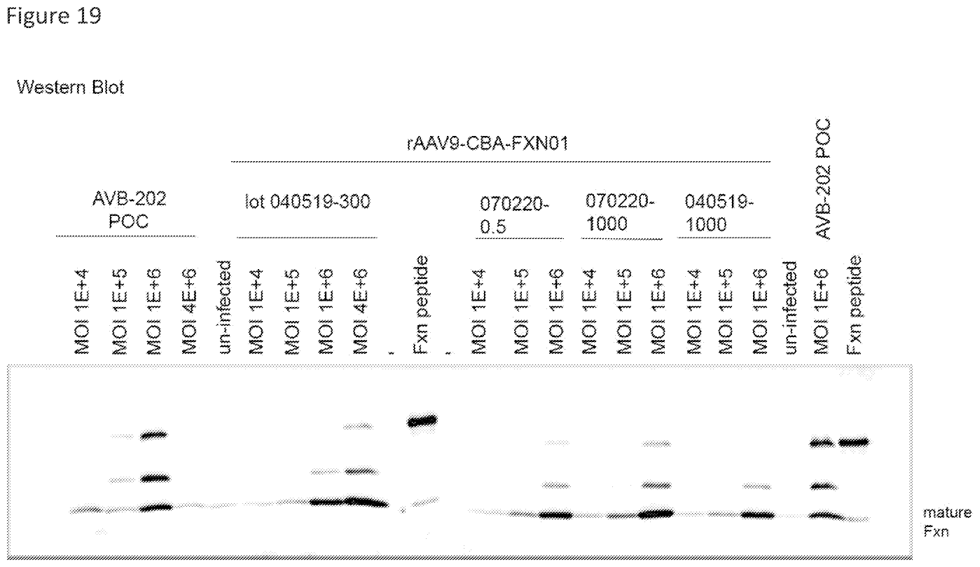

In an additional experiment, the qualitative levels of expression between the frataxin gene utilized in the experiments and data represented in FIGS. 1-15 and 17-18 (“FXN01”; SEQ ID NO: 11) and the improved codon-optimized frataxin gene (“AVB-202”; SEQ ID NO: 1, SEQ ID NO: 2) was evaluated. FIG. 19 shows expression of each of the expected frataxin forms (full length, intermediate, and mature forms, which arise following variations in post-translational proteolytic processing) when HEK293 cells were infected with rAAV at the multiplicity of infections (MOI) noted. rAAV comprising the improved codon-optimized frataxin gene (“AVB-202”; SEQ ID NO: 1, SEQ ID NO: 2) resulted in a qualitative increase in expression as compared to rAAV comprising SEQ ID NO: 11 (lanes labeled “rAAV9-CBA-FXN01”).

Taken together, these experiments show that frataxin is successfully expressed and process in a durable fashion. It is also expressed in several of the target organs implicated in the symptoms and progression of FA, namely the heart and brain. In several embodiments, therefore, administration of a viral vector encoding human frataxin, such as the codon-optimized human frataxin disclosed herein can successfully reduce or even prevent the progression of FA in patients.

Minimal Pathology with Viral-based Frataxin Delivery

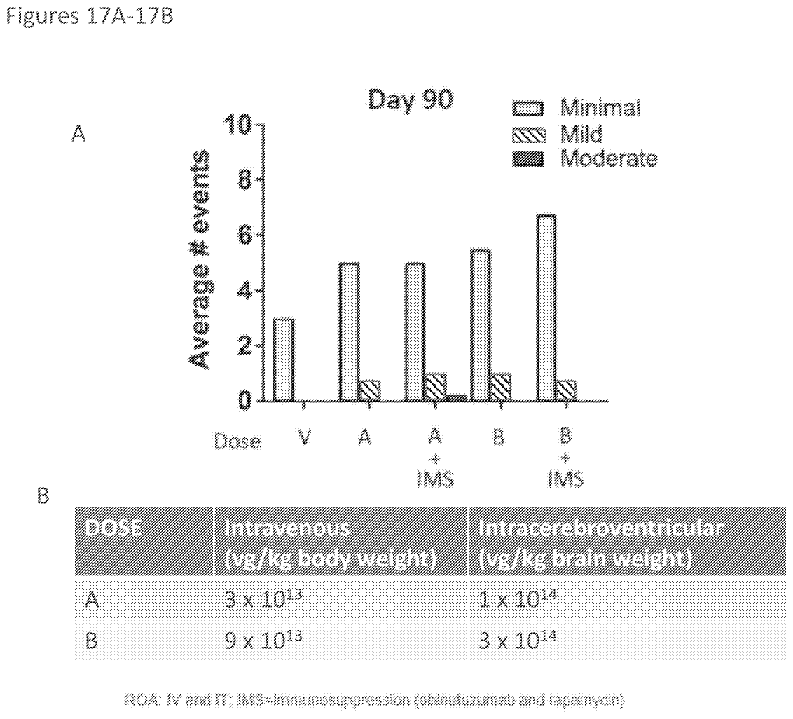

The successful demonstration of functional benefits of frataxin and ability of frataxin to be expressed are met with the next hurdle, that is whether delivery of a viral vector encoding human frataxin may result in any adverse pathologies. FIG. 17A (doses in FIG. 17B) show an assessment of the number of adverse pathologies identified 90 days after viral vector administration to non-human primates. Vectors were administered with or without an immunosuppression regimen of obinutuzumab and rapamycin. The dorsal root ganglia and spinal cord were assessed for histopathologies. FIG. 17A summarizes that the vast majority of histopathologies were minimal in nature. The data show that mild histopathologies were more infrequent with moderate being rare/non-existent. At the higher doses, it appears that there is a minimal increase in minimal histopathologies, and that immunosuppression may have some beneficial reduction in mild pathologies.

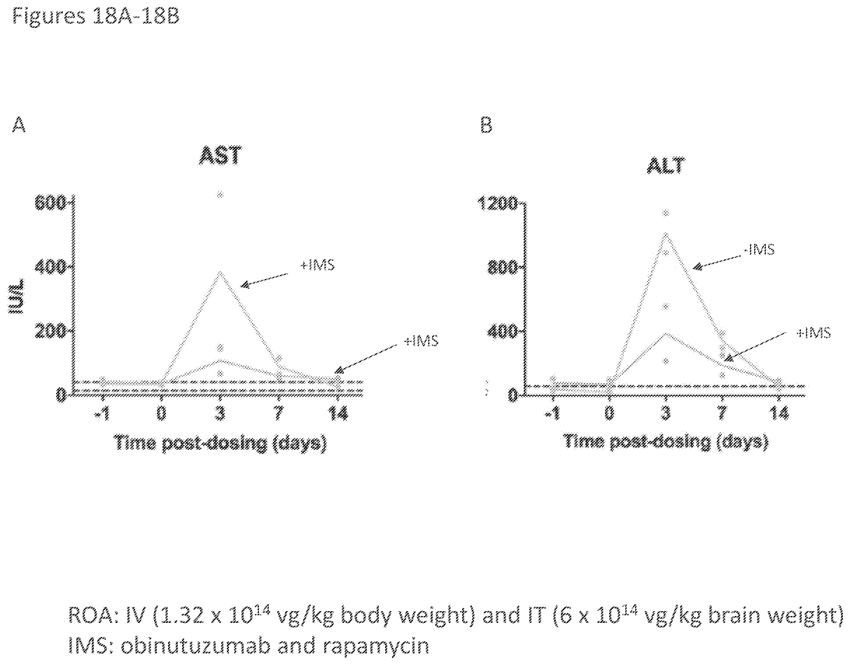

Administration of high-dose rAAV-hFXN to non-human primates with an immunosuppression regimen had a very mild impact on liver function. rAAV-FXN was administered either intravenously (IV) at a dose of 1.32×10¹⁴ vg/kg body weight, or intrathecally (IT) at a dose of 6×10¹⁴ vg/kg brain weight. FIG. 18A shows data related to the detection of aspartate transaminase (AST) in the blood of non-human primates after receiving high-dose rAAV-hFXN either with, or without, the immunosuppression (IMS) regimen of obinutuzumab and rapamycin. FIG. 18B shows data related to the detection of alanine aminotransferase (ALT) in the blood of non-human primates after receiving high-dose rAAV-hFXN either with, or without, the immunosuppression regimen. As shown in FIGS. 18A and 18B, the largest effect on liver function was observed at 3 days post-dosing, both with and without IMS. Addition of IMS reduced liver enzyme activity by about 60-75% following IV and IT administrations. This data indicates that co-administration of an immunosuppression regimen should moderate effects in the liver from vector administration.

### EQUIVALENTS

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% homologous includes 96%, 97%, 98%, 99%, and 100% homologous to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

In several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

Any titles or subheadings used herein are for organization purposes and should not be used to limit the scope of embodiments disclosed herein.

What is claimed is:

1. A polynucleotide encoding a codon optimized human frataxin, comprising a DNA sequence that has been codon optimized for enhanced expression and/or function in human cells having a sequence that comprises at least 85%, at least 90%, at least 95%, at least 97%, at least 98%, at least 99%, or more sequence identity to SEQ ID NO: 1 or SEQ ID NO: 2.

2. The polynucleotide of claim 1, wherein the polynucleotide comprises a sequence having at least 99%, or more sequence identity to SEQ ID NO: 1 or SEQ ID NO: 2.

3. The polynucleotide of claim 1 or 2, wherein the polynucleotide encodes human frataxin comprising the amino acid sequence of SEQ ID NO: 3.

4. The polynucleotide of any one of claims 1-3, wherein the polynucleotide exhibits enhanced expression of human frataxin as compared to a polynucleotide encoding frataxin but not having the same sequence identity to SEQ ID NO: 1 or SEQ ID NO: 2.

5. A polynucleotide of any one of claims 1-4, wherein the polynucleotide further comprises an untranslated region (UTR) that imparts regulatory control on expression of the human frataxin encoded by the polynucleotide.

6. The polynucleotide of claim 5, wherein the untranslated region comprises a sequence that comprises at least 85%, at least 90%, at least 95%, at least 97%, at least 98%, at least 99%, or more sequence identity to SEQ ID NO: 6.

7. A polynucleotide of any one of claims 1-6, wherein the polynucleotide further comprises a promoter driving expression of the frataxin.

8. The polynucleotide of claim 7, wherein the promoter comprises a cytomegalovirus enhancer element functionally coupled to a chicken beta actin promoter.

9. The polynucleotide of claim 8, wherein the cytomegalovirus enhancer element comprises a sequence that comprises at least 85%, at least 90%, at least 95%, at least 97%, at least 98%, at least 99%, or more sequence identity to SEQ ID NO: 4.

10. The polynucleotide of claim 8 or 9, wherein the chicken beta actin promoter comprises a sequence that comprises at least 85%, at least 90%, at least 95%, at least 97%, at least 98%, at least 99%, or more sequence identity to SEQ ID NO: 5.

11. A plasmid encoding the polynucleotide of any one of claims 1-10, wherein the plasmid is at least 90% identical to SEQ ID NO: 8.

12. A recombinant adeno-associated virus (rAAV) vector for use in treating Friedreich's ataxia, wherein the vector comprises an expression construct comprising a promoter, a transgene encoding human frataxin, and a 3′ untranslated region, wherein the transgene encoding human frataxin comprises a DNA sequence that has been codon optimized for enhanced expression and/or function in human cells comprising a sequence that comprises at least 85%, at least 90%, at least 95%, at least 97%, at least 98%, at least 99%, or more sequence identity to SEQ ID NO: 1 or SEQ ID NO: 2.

13. The rAAV of claim 12, wherein the rAAV is serotype 9.

14. The rAAV of claim 12, wherein the rAAV is serotype rh74.

15. The rAAV of any one of claims 12-14, wherein the rAAV comprises the polynucleotide of any one of claims 1 to 10.

16. A system for treating Friedreich's Ataxia comprising an rAAV vector according to any one of claims 12 to 15, and an immunomodulatory regimen.

17. The system of claim 16, wherein the immunomodulatory regimen comprises an antibody directed against a cancer marker and an additional agent.

18. The system of claim 17, wherein the cancer marker is CD20.

19. The system of claim 17 or 18, wherein the additional agent is an mTOR pathway inhibitor.

20. A dosing regimen for treatment of Friedreich's Ataxia, wherein the regimen comprises a first dose for intravenous delivery to a subject, and a second dose for delivery to the cerebrospinal fluid of the subject.

21. The dosing regimen of claim 20, wherein the amount of the first dose and the amount of the second dose are within about 1 to about 20 times one another.

22. The dosing regimen of claim 20 or 21, wherein the first dose for intravenous delivery ranges from about 1×10¹³ to about 9×10¹⁵ viral genomes per kg body weight of a subject, and the second dose for delivery to the cerebrospinal fluid ranges from about 9×10¹³ to about 9×10¹⁵ viral genomes per kg brain weight of the subject.

23. The dosing regimen of any one of claims 20-22, wherein the second dose is delivered intrathecally.

24. The dosing regimen of any one of claims 20-23, further comprising an immunomodulatory regimen.

25. The dosing regimen of claim 24, wherein the immunomodulatory regimen comprises an antibody directed against a cancer marker and an additional agent.

26. The dosing regimen of claim 25, wherein the cancer marker is CD20.

27. The dosing regimen of claim 25 or 26, wherein the additional agent is an mTOR pathway inhibitor.

28. A method of treating Friedreich's Ataxia in a subject in need thereof, comprising:
administering to the subject a first dose of an rAAV vector encoding codon optimized human frataxin,
wherein the codon optimized human frataxin comprises a DNA sequence that has been codon optimized for enhanced expression and/or function in human cells comprising a sequence that comprises at least 85%, at least 90%, at least 95%, at least 97%, at least 98%, at least 99%, or more sequence identity to SEQ ID NO: 1 or SEQ ID NO: 2,
wherein the first administration is intravenous, and
administering to the subject a second dose of the rAAV vector encoding the codon optimized human frataxin, and
wherein the second administration is to the cerebrospinal fluid.

29. The method of claim 28, wherein the rAAV vector is serotype 9 or rh74.

30. The method of claim 28 or 29 further comprising administration of an immunomodulatory regimen comprising an antibody directed against a cancer marker and an additional agent.

31. The method of claim 30, wherein the cancer marker is CD20.

32. The method of claim 30 or 31, wherein the additional agent is an mTOR pathway inhibitor.

33. The method of any one of claims 30-32, wherein the immunomodulatory regimen is administered prior to the rAAV.

34. The method of any one of claims 30-33, wherein the steps of administering the first dose and the second dose provides improved cardiac function in the subject.

35. The method of any one of claims 30-34, wherein the steps of administering the first dose and the second dose provides improved maintenance of body weight and/or muscle mass in the subject.

36. The method of any one of claims 30-35, wherein the subject is human.

37. Use of the polynucleotide of any one of claims 1-10, the plasmid of claim 11, the raav of claims 12-15, the system of claim 16-19 or the dosing regimen of claims 20-27 for treating Friedreich's ataxia.

38. Use of the polynucleotide of any one of claims 1-10, the plasmid of claim 11, the raav of claims 12-15, the system of claim 16-19 or the dosing regimen of claims 20-27 for the manufacture of a medicament for treating Friedreich's ataxia.


SEQUENCE LISTING (12 sequences)

SEQ ID NO: 1 - DNA, 633 bp, Homo sapiens
  misc_feature: hFXN codon opt (w/ stop codon)
atgtggaccctcggccgcagagctgttgctggacttcttgcctctccatctccagctcaggcccagacactgaccagagtgcctagacctgctgaactggcccctctgtgtggcagaagaggcctgagaaccgacatcgacgccacatgcacacctagaagggccagcagcaatcagcggggcctgaatcagatctggaacgtgaagaaacagagcgtgtacctgatgaacctgagaaagagcggcaccctgggacaccctggaagcctggatgagacaacctacgagagactggccgaggaaaccctggattccctggccgagttcttcgaggacctggccgataagccctacaccttcgaggattacgacgtgtcctttggcagcggcgtgctgacagtgaaactcggcggagatctgggcacctacgtgatcaacaagcagacccctaacaaacagatctggctgagcagccctagcagcggccccaagagatatgattggaccggcaagaactgggtgtacagccacgatggcgtgtccctgcacgaactgctggctgccgaactgacaaaggccctgaaaacaaagctggacctgtccagcctggcctactctggcaaggacgcctga

SEQ ID NO: 2 - DNA, 630 bp, Homo sapiens
  misc_feature: hFXN codon optimized (no stop codon)
atgtggaccctcggccgcagagctgttgctggacttcttgcctctccatctccagctcaggcccagacactgaccagagtgcctagacctgctgaactggcccctctgtgtggcagaagaggcctgagaaccgacatcgacgccacatgcacacctagaagggccagcagcaatcagcggggcctgaatcagatctggaacgtgaagaaacagagcgtgtacctgatgaacctgagaaagagcggcaccctgggacaccctggaagcctggatgagacaacctacgagagactggccgaggaaaccctggattccctggccgagttcttcgaggacctggccgataagccctacaccttcgaggattacgacgtgtcctttggcagcggcgtgctgacagtgaaactcggcggagatctgggcacctacgtgatcaacaagcagacccctaacaaacagatctggctgagcagccctagcagcggccccaagagatatgattggaccggcaagaactgggtgtacagccacgatggcgtgtccctgcacgaactgctggctgccgaactgacaaaggccctgaaaacaaagctggacctgtccagcctggcctactctggcaaggacgcc

SEQ ID NO: 3 - protein, 210 aa, Homo sapiens
  MISC_FEATURE: hFXN codon optimized AA
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 4 - DNA, 367 bp, Artificial Sequence
  Synthetic polynucleotide
  misc_feature: CMV enchancer of CBA
ccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 5 - DNA, 278 bp, Artificial Sequence
  Synthetic polynucleotide
  misc_feature: chicken beta actin promoter
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 6 - DNA, 1813 bp, Artificial Sequence
  Synthetic polynucleotide
  misc_feature: 3′-UTR
tcgagtgccccagccccgttttaaggacattaaaagctatcaggccaagaccccagcttcattatgcagctgaggtctgttttttgttgttgttgttgtttattttttttattcctgcttttgaggacagttgggctatgtgtcacagctctgtagaaagaatgtgttgcctcctaccttgcccccaagttctgatttttaatttctatggaagattttttggattgtcggatttcctccctcacatgataccccttatcttttataatgtcttatgcctatacctgaatataacaacctttaaaaaagcaaaataataagaaggaaaaattccaggagggaaaatgaattgtcttcactcttcattctttgaaggatttactgcaagaagtacatgaagagcagctggtcaacctgctcactgttctatctccaaatgagacacattaaagggtagcctacaaatgttttcaggcttctttcaaagtgtaagcacttctgagctctttagcattgaagtgtcgaaagcaactcacacgggaagatcatttcttatttgtgctctgtgactgccaaggtgtggcctgcactgggttgtccagggagacatgcatctagtgctgtttctcccacatattcacatacgtgtctgtgtgtatatatattttttcaatttaaaggttagtatggaatcagctgctacaagaatgcaaaaaatcttccaaagacaagaaaagaggaaaaaaagccgttttcatgagctgagtgatgtagcgtaacaaacaaaatcatggagctgaggaggtgccttgtaaacatgaaggggcagataaaggaaggagatactcatgttgataaagagagccctggtcctagacatagttcagccacaaagtagttgtccctttgtggacaagtttcccaaattccctggacctctgcttccccatctgttaaatgagagaatagagtatggttgattcccagcattcagtggtcctgtcaagcaacctaacaggctagttctaattccctattgggtagatgaggggatgacaaagaacagtttttaagctatataggaaacattgttattggtgttgccctatcgtgatttcagttgaattcatgtgaaaataatagccatccttggcctggcgcggtggctcacacctgtaatcccagcacttttggaggccaaggtgggtggatcacctgaggtcaggagttcaagaccagcctggccaacatgatgaaaccccgtctctactaaaaatacaaaaaattagccgggcatgatggcaggtgcctgtaatcccagctacttgggaggctgaagcggaagaatcgcttgaacccagaggtggaggttgcagtgagccgagatcgtgccattgcactgtaacctgggtgactgagcaaaactctgtctcaaaataataataacaatataataataataatagccatcctttattgtacccttactgggttaatcgtattataccacattacctcattttaatttttactgacctgcactttatacaaagcaacaagcctccaggacattaaaattcatgcaaagttatgctcatgttatattattttcttacttaaagaaggatttattagtggctgggcatggtggcgtgcacctgtaatcccaggtactcaggaggctgagacgggagaattgcttgaccccaggcggaggaggttacagtgagtcgagatcgtacctgagcgacagagcgagactccgtctcaaaaaaaaaaaaaaggagggtttattaatgagaagtttggtcga

SEQ ID NO: 7 - DNA, 1019 bp, Artificial Sequence
  Synthetic polynucleotide
  misc_feature: chimeric intron
ggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 8 - DNA, 7637 bp, Artificial Sequence
  Synthetic polynucleotide
  misc_feature: B757_pTR_CBA_hFXNco_3′UTR plasmid
ctgcaggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatccgaaggggttcaagcttaaaaactagtgccaccatgtggaccctcggccgcagagctgttgctggacttcttgcctctccatctccagctcaggcccagacactgaccagagtgcctagacctgctgaactggcccctctgtgtggcagaagaggcctgagaaccgacatcgacgccacatgcacacctagaagggccagcagcaatcagcggggcctgaatcagatctggaacgtgaagaaacagagcgtgtacctgatgaacctgagaaagagcggcaccctgggacaccctggaagcctggatgagacaacctacgagagactggccgaggaaaccctggattccctggccgagttcttcgaggacctggccgataagccctacaccttcgaggattacgacgtgtcctttggcagcggcgtgctgacagtgaaactcggcggagatctgggcacctacgtgatcaacaagcagacccctaacaaacagatctggctgagcagccctagcagcggccccaagagatatgattggaccggcaagaactgggtgtacagccacgatggcgtgtccctgcacgaactgctggctgccgaactgacaaaggccctgaaaacaaagctggacctgtccagcctggcctactctggcaaggacgcctgatgatgactcgagtgccccagccccgttttaaggacattaaaagctatcaggccaagaccccagcttcattatgcagctgaggtctgttttttgttgttgttgttgtttattttttttattcctgcttttgaggacagttgggctatgtgtcacagctctgtagaaagaatgtgttgcctcctaccttgcccccaagttctgatttttaatttctatggaagattttttggattgtcggatttcctccctcacatgataccccttatcttttataatgtcttatgcctatacctgaatataacaacctttaaaaaagcaaaataataagaaggaaaaattccaggagggaaaatgaattgtcttcactcttcattctttgaaggatttactgcaagaagtacatgaagagcagctggtcaacctgctcactgttctatctccaaatgagacacattaaagggtagcctacaaatgttttcaggcttctttcaaagtgtaagcacttctgagctctttagcattgaagtgtcgaaagcaactcacacgggaagatcatttcttatttgtgctctgtgactgccaaggtgtggcctgcactgggttgtccagggagacatgcatctagtgctgtttctcccacatattcacatacgtgtctgtgtgtatatatattttttcaatttaaaggttagtatggaatcagctgctacaagaatgcaaaaaatcttccaaagacaagaaaagaggaaaaaaagccgttttcatgagctgagtgatgtagcgtaacaaacaaaatcatggagctgaggaggtgccttgtaaacatgaaggggcagataaaggaaggagatactcatgttgataaagagagccctggtcctagacatagttcagccacaaagtagttgtccctttgtggacaagtttcccaaattccctggacctctgcttccccatctgttaaatgagagaatagagtatggttgattcccagcattcagtggtcctgtcaagcaacctaacaggctagttctaattccctattgggtagatgaggggatgacaaagaacagtttttaagctatataggaaacattgttattggtgttgccctatcgtgatttcagttgaattcatgtgaaaataatagccatccttggcctggcgcggtggctcacacctgtaatcccagcacttttggaggccaaggtgggtggatcacctgaggtcaggagttcaagaccagcctggccaacatgatgaaaccccgtctctactaaaaatacaaaaaattagccgggcatgatggcaggtgcctgtaatcccagctacttgggaggctgaagcggaagaatcgcttgaacccagaggtggaggttgcagtgagccgagatcgtgccattgcactgtaacctgggtgactgagcaaaactctgtctcaaaataataataacaatataataataataatagccatcctttattgtacccttactgggttaatcgtattataccacattacctcattttaatttttactgacctgcactttatacaaagcaacaagcctccaggacattaaaattcatgcaaagttatgctcatgttatattattttcttacttaaagaaggatttattagtggctgggcatggtggcgtgcacctgtaatcccaggtactcaggaggctgagacgggagaattgcttgaccccaggcggaggaggttacagtgagtcgagatcgtacctgagcgacagagcgagactccgtctcaaaaaaaaaaaaaaggagggtttattaatgagaagtttggtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctgg

SEQ ID NO: 9 - DNA, 712 bp, Homo sapiens
  misc_feature: Desmin promoter
gatcttaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgagataaccagggctgaaagaggcccgcctgggggctggagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgtccgccgcctcctccgtgcgcccgccagcctcgcccgcgccgtcaccgtgaggcactggg

SEQ ID NO: 10 - DNA, 1220 bp, Homo sapiens
  misc_feature: human frataxin promoter
aagaaaactttcacaatttgcatccctttgtaatatgtaacagaaataaaattctcttttaaaatctatcaacaataggcaaggcacggtggctcacgcctgtcgtctcagcactttgtgaggcccaggcgggcagatcgtttgagcctagaagttcaagaccaccctgggcaacatagcgaaaccccctttctacaaaaaatacaaaaactagctgggtgtggtggtgcacacctgtagtcccagctacttggaaggctgaaatgggaagactgcttgagcccgggagggagaagttgcagtaagccaggaccacaccactgcactccagcctgggcaacagagtgagactctgtctcaaacaaacaaataaatgaggcgggtggatcacgaggtcagtagatcgagaccatcctggctaacacggtgaaacccgtctctactaaaaaaaaaaaaaaatacaaaaaattagccaggcatggtggcgggcgcctgtagtcccagttactcgggaggctgaggcaggagaatggcgtgaaaccgggaggcagagcttgcagtgagccgagatcgcaccactgccctccagcctgggcgacagagcgagactccgtctcaatcaatcaatcaatcaataaaatctattaacaatatttattgtgcacttaacaggaacatgccctgtccaaaaaaaactttacagggcttaactcattttatccttaccacaatcctatgaagtaggaacttttataaaacgcattttataaacaaggcacagagaggttaattaacttgccctctggtcacacagctaggaagtgggcagagtacagatttacacaaggcatccgtctcctggccccacatacccaactgctgtaaacccataccggcggccaagcagcctcaatttgtgcatgcacccacttcccagcaagacagcagctcccaagttcctcctgtttagaattttagaagcggcgggccaccaggctgcagtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagc

SEQ ID NO: 11 - DNA, 630 bp, Homo sapiens
atgtggacactggggagaagggccgtggctggactgctggcttctccatctccagcccaggcccagaccctgaccagagtgcctagacctgccgaactggcccctctgtgtggcagaagaggcctgagaaccgacatcgacgccacctgtacccccagaagggccagcagcaatcagcggggcctgaatcagatctggaacgtgaagaaacagagcgtgtacctgatgaacctgagaaagagcggcaccctgggccaccctggaagcctggatgagacaacctacgagcggctggccgaggaaaccctggattccctggccgagttcttcgaggacctggccgacaagccctacaccttcgaggattacgacgtgtccttcggcagcggcgtgctgacagtgaagctgggcggagatctgggcacctacgtgatcaacaagcagacccccaacaaacagatctggctgagcagccccagcagcggccccaagagatacgattggaccggcaagaactgggtgttcagccacgacggcgtgtccctgcatgagctgctggctgccgagctgaccaaggccctgaaaacaaagctggacctgagctggctggcctacagcggcaaagatgcc

SEQ ID NO: 12 - DNA, 1490 bp, Artificial Sequence
  Synthetic
aagaaggaaaaattccaggagggaaaatgaattgtcttcactcttcattctttgaaggatttactgcaagaagtacatgaagagcagctggtcaacctgctcactgttctatctccaaatgagacacattaaagggtagcctacaaatgttttcaggcttctttcaaagtgtaagcacttctgagctctttagcattgaagtgtcgaaagcaactcacacgggaagatcatttcttatttgtgctctgtgactgccaaggtgtggcctgcactgggttgtccagggagacatgcatctagtgctgtttctcccacatattcacatacgtgtctgtgtgtatatatattttttcaatttaaaggttagtatggaatcagctgctacaagaatgcaaaaaatcttccaaagacaagaaaagaggaaaaaaagccgttttcatgagctgagtgatgtagcgtaacaaacaaaatcatggagctgaggaggtgccttgtaaacatgaaggggcagataaaggaaggagatactcatgttgataaagagagccctggtcctagacatagttcagccacaaagtagttgtccctttgtggacaagtttcccaaattccctggacctctgcttccccatctgttaaatgagagaatagagtatggttgattcccagcattcagtggtcctgtcaagcaacctaacaggctagttctaattccctattgggtagatgaggggatgacaaagaacagtttttaagctatataggaaacattgttattggtgttgccctatcgtgatttcagttgaattcatgtgaaaataatagccatccttggcctggcgcggtggctcacacctgtaatcccagcacttttggaggccaaggtgggtggatcacctgaggtcaggagttcaagaccagcctggccaacatgatgaaaccccgtctctactaaaaatacaaaaaattagccgggcatgatggcaggtgcctgtaatcccagctacttgggaggctgaagcggaagaatcgcttgaacccagaggtggaggttgcagtgagccgagatcgtgccattgcactgtaacctgggtgactgagcaaaactctgtctcaaaataataataacaatataataataataatagccatcctttattgtacccttactgggttaatcgtattataccacattacctcattttaatttttactgacctgcactttatacaaagcaacaagcctccaggacattaaaattcatgcaaagttatgctcatgttatattattttcttacttaaagaaggatttattagtggctgggcatggtggcgtgcacctgtaatcccaggtactcaggaggctgagacgggagaattgcttgaccccaggcggaggaggttacagtgagtcgagatcgtacctgagcgacagagcgagactccgtctcaaaaaaaaaaaaaaggagggtttattaatgagaagtttg